(19) United States
(12) Patent Application Publication
(10) Pub. No.: US 2024/0327419 A1
(43) Pub. Date: Oct. 3, 2024

(54) SYNTHESIS OF SUBSTITUTED TRICYCLIC AMIDES AND ANALOGUES THEREOF

(71) Applicant: ARBUTUS BIOPHARMA CORPORATION, Vancouver (CA)

(72) Inventors: Jeremy Mason, Warminster, PA (US); Mahesh Pallerla, Chalfont, PA (US); Ganapati Reddy Pamulapati, Warrington, PA (US)

(21) Appl. No.: 18/578,467

(22) PCT Filed: Jul. 15, 2022
(86) PCT No.: PCT/IB2022/056546

§ 371 Date: Jan. 11, 2024

Related U.S. Application Data
(60) Provisional application No. 63/223,297, filed on Jul. 19, 2021.

(51) Int. Cl. C07D 491/052; C07D 209/12; C07D 493/04

(52) U.S. Cl. CPC C07D 491/052; C07D 209/12; C07D 493/04

(57) ABSTRACT

The present disclosure includes synthetic methods for preparing certain substituted tricyclic amides, which can be used to treat, ameliorate, and/or prevent hepatitis B virus (HBV) and/or hepatitis D virus (HDV) infections in a subject.

### CROSS-REFERENCE TO RELATED APPLICATIONS

The present application claims priority under 35 U.S.C. § 119(e) to U.S. Provisional Patent Application No. 63/223,297, filed Jul. 19, 2021, which is hereby incorporated herein by reference in its entirety.

### BACKGROUND

Hepatitis B is one of the world's most prevalent diseases, being listed by National Institute of Allergy and Infectious Diseases (NIAID) as a High Priority Area of Interest. Although most individuals resolve the infection following acute symptoms, approximately 30% of cases become chronic. 350-400 million people worldwide are estimated to have chronic hepatitis B, leading to 0.5-1 million deaths per year, due largely to the development of hepatocellular carcinoma, cirrhosis and/or other complications.

A limited number of drugs are currently approved for the management of chronic hepatitis B, including two formulations of alpha-interferon (standard and pegylated) and five nucleoside/nucleotide analogues (lamivudine, adefovir, entecavir, telbivudine, and tenofovir) that inhibit hepatitis B virus (HBV) DNA polymerase. At present, the first-line treatment choices are entecavir, tenofovir and/or peg-interferon alfa-2a. However, peg-interferon alfa-2a achieves desirable serological milestones in only one third of treated patients, and is frequently associated with severe side effects. Entecavir and tenofovir are potent HBV inhibitors, but require long-term or possibly lifetime administration to continuously suppress HBV replication, and may eventually fail due to emergence of drug-resistant viruses. There is thus a pressing need for the introduction of novel, safe, and effective therapies for chronic hepatitis B.

HBV is a noncytopathic, liver tropic DNA virus belonging to Hepadnaviridae family. Pregenomic (pg) RNA is the template for reverse transcriptional replication of HBV DNA. The encapsidation of pg RNA, together with viral DNA polymerase, into a nucleocapsid is essential for the subsequent viral DNA synthesis. Inhibition of pg RNA encapsidation may block HBV replication and provide a new therapeutic approach to HBV treatment. A capsid inhibitor acts by inhibiting the expression and/or function of a capsid protein either directly or indirectly: for example, it may inhibit capsid assembly, induce formation of non-capsid polymers, promote excess capsid assembly or misdirected capsid assembly, affect capsid stabilization, and/or inhibit RNA encapsidation. A capsid inhibitor may also act by inhibiting capsid function in one or more downstream events within the replication process, such as, but not limited to, viral DNA synthesis, transport of relaxed circular DNA (rcDNA) into the nucleus, covalently closed circular DNA (cccDNA) formation, virus maturation, budding and/or release.

Clinically, inhibition of pg RNA encapsidation, or more generally inhibition of nucleocapsid assembly, may offer certain therapeutic advantages. In one aspect, inhibition of pg RNA encapsidation may complement the current medications by providing an option for a subpopulation of patients that do not tolerate or benefit from the current medications. In another aspect, based on their distinct antiviral mechanism, inhibition of pg RNA encapsidation may be effective against HBV variants resistant to the currently available DNA polymerase inhibitors. In yet another aspect, combination therapy of the pg RNA encapsidation inhibitors with DNA polymerase inhibitors may synergistically suppress HBV replication and prevent drug resistance emergence, thus offering a more effective treatment for chronic hepatitis B infection.

Hepatitis D virus (HDV) is a small circular enveloped RNA virus that can propagate only in the presence of HBV. In particular, HDV requires the HBV surface antigen protein to propagate itself. Infection with both HBV and HDV results in more severe complications compared to infection with HBV alone. These complications include a greater likelihood of experiencing liver failure in acute infections and a rapid progression to liver cirrhosis, with an increased chance of developing liver cancer in chronic infections. In combination with hepatitis B, hepatitis D has the highest mortality rate of all the hepatitis infections. The routes of transmission of HDV are similar to those for HBV. Infection is largely restricted to persons at high risk of HBV infection, particularly injecting drug users and persons receiving clotting factor concentrates.

Currently, there is no effective antiviral therapy available for the treatment of acute or chronic type D hepatitis. Interferon-alfa given weekly for 12 to 18 months is the only licensed treatment for hepatitis D. Response to this therapy is limited, as only about one-quarter of patients is serum HDV RNA undetectable 6 months post therapy.

Clinically, inhibition of pg RNA encapsidation, or more generally inhibition of nucleocapsid assembly, may offer certain therapeutic advantages for treatment of hepatitis B and/or hepatitis D. In one aspect, inhibition of pg RNA encapsidation may complement the current medications by providing an option for a subpopulation of patients that do not tolerate or benefit from the current medications. In another aspect, based on their distinct antiviral mechanism, inhibition of pg RNA encapsidation may be effective against HBV and/or HDV variants resistant to the currently available DNA polymerase inhibitors. In yet another aspect, combination therapy of the pg RNA encapsidation inhibitors with DNA polymerase inhibitors may synergistically suppress HBV and/or HDV replication and prevent drug resistance emergence, thus offering a more effective treatment for chronic hepatitis B and/or hepatitis D infection.

There is thus a need in the art for the identification of novel compounds that can be used to treat and/or prevent HBV and/or HDV infection in a subject. In certain embodiments, the novel compounds inhibit HBV and/or HDV nucleocapsid assembly. In other embodiments, the novel compounds can be used in patients that are HBV and/or HBV-HDV infected, patients who are at risk of becoming HBV and/or HBV-HDV infected, and/or patients that are infected with drug-resistant HBV and/or HDV. There is a further need in the art to identify scalable synthetic schemes that allow for the preparation of large scale batches of such compounds. The present disclosure addresses this need.

### BRIEF SUMMARY

The present disclosure includes, in one aspect, methods of preparing (S)—N-(8,9-difluoro-6-oxo-1,4,5,6-tetrahydro-2H-pyrano[3,4-c]isoquinolin-1-yl)-5,6-difluoro-N-methyl-1H-indole-2-carboxamide (X), or a salt, solvate, prodrug, isotopically labeled derivative, stereoisomer (such as in a non-limiting example, an enantiomer or any mixtures thereof, such as, in a non-limiting example, mixtures in any proportions of enantiomers thereof), and/or tautomer, and any mixtures thereof:

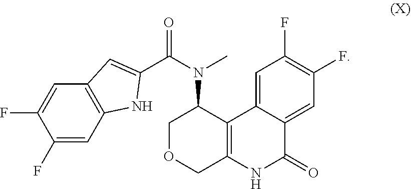 [SMILES: CN(C(=O)c1cc2cc(F)c(F)cc2[nH]1)[C@@H]1COCc2[nH]c(=O)c3cc(F)c(F)cc3c21]

In another aspect, the compound of formula (X), or a salt, solvate, prodrug, isotopically labeled derivative, stereoisomer, and/or tautomer, and any mixtures thereof, is useful to treat, ameliorate, and/or prevent hepatitis B (HBV) and/or hepatitis D (HDV) infection and related conditions in a subject.

### DETAILED DESCRIPTION OF THE DISCLOSURE

The disclosure relates, in certain aspects, to the discovery of scalable synthetic routes that allow for reproducible multi-gram synthesis of certain substituted tricyclic amide-containing compounds that are useful to treat, ameliorate, and/or prevent hepatitis B virus (HBV) and/or hepatitis D virus (HDV) infection and related conditions in a subject. In certain embodiments, the compounds of the disclosure are viral capsid inhibitors.

The disclosures of PCT International Application No. PCT/US2021/032155 filed May 13, 2021 and U.S. Provisional Application No. 63/024,559 filed May 14, 2020 are incorporated herein by reference in their entireties.

### Definitions

As used herein, each of the following terms has the meaning associated with it in this section. Unless defined otherwise, all technical and scientific terms used herein generally have the same meaning as commonly understood by one of ordinary skill in the art to which this disclosure belongs. Generally, the nomenclature used herein and the laboratory procedures in animal pharmacology, pharmaceutical science, separation science, and organic chemistry are those well-known and commonly employed in the art. It should be understood that the order of steps or order for performing certain actions is immaterial, so long as the present teachings remain operable. Any use of section headings is intended to aid reading of the document and is not to be interpreted as limiting; information that is relevant to a section heading may occur within or outside of that particular section. All publications, patents, and patent documents referred to in this document are incorporated by reference herein in their entirety, as though individually incorporated by reference.

In the application, where an element or component is said to be included in and/or selected from a list of recited elements or components, it should be understood that the element or component can be any one of the recited elements or components and can be selected from a group consisting of two or more of the recited elements or components.

In the methods described herein, the acts can be carried out in any order, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.”

As used herein, the term “about” will be understood by persons of ordinary skill in the art and will vary to some extent on the context in which it is used. As used herein, “about” when referring to a measurable value such as an amount, a temporal duration, and the like, is meant to encompass variations of +20%, +10%, +5%, +1%, or +0.1% from the specified value, as such variations are appropriate to perform the disclosed methods.

As used herein, the term “alkenyl,” employed alone or in combination with other terms, means, unless otherwise stated, a stable monounsaturated or diunsaturated straight chain or branched chain hydrocarbon group having the stated number of carbon atoms. Examples include vinyl, propenyl (or allyl), crotyl, isopentenyl, butadienyl, 1,3-pentadienyl, 1,4-pentadienyl, and the higher homologs and isomers. A functional group representing an alkene is exemplified by —CH₂—CH═CH₂.

As used herein, the term “alkoxy” employed alone or in combination with other terms means, unless otherwise stated, an alkyl group having the designated number of carbon atoms, as defined elsewhere herein, connected to the rest of the molecule via an oxygen atom, such as, for example, methoxy, ethoxy, 1-propoxy, 2-propoxy (or isopropoxy) and the higher homologs and isomers. A specific example is (C₁-C₃)alkoxy, such as, but not limited to, ethoxy and methoxy.

As used herein, the term “alkyl” by itself or as part of another substituent means, unless otherwise stated, a straight or branched chain hydrocarbon having the number of carbon atoms designated (i.e., C₁-C₁₀ means one to ten carbon atoms) and includes straight, branched chain, or cyclic substituent groups. Examples include methyl, ethyl, propyl, isopropyl, butyl, isobutyl, tert-butyl, pentyl, neopentyl, hexyl, and cyclopropylmethyl. A specific embodiment is (C₁-C₆)alkyl, such as, but not limited to, ethyl, methyl, isopropyl, isobutyl, n-pentyl, n-hexyl, and cyclopropylmethyl.

As used herein, the term “alkynyl” employed alone or in combination with other terms means, unless otherwise stated, a stable straight chain or branched chain hydrocarbon group with a triple carbon-carbon bond, having the stated number of carbon atoms. Non-limiting examples include ethynyl and propynyl, and the higher homologs and isomers. The term “propargylic” refers to a group exemplified by —CH₂—C≡CH. The term “homopropargylic” refers to a group exemplified by —CH₂CH₂—C≡CH.

As used herein, the term “aromatic” refers to a carbocycle or heterocycle with one or more polyunsaturated rings and having aromatic character, i.e., having (4n+2) delocalized π (pi) electrons, where ‘n’ is an integer.

As used herein, the term “aryl” employed alone or in combination with other terms means, unless otherwise stated, a carbocyclic aromatic system containing one or more rings (typically one, two or three rings) wherein such rings may be attached together in a pendent manner, such as a biphenyl, or may be fused, such as naphthalene. Examples include phenyl, anthracyl and naphthyl. Aryl groups also include, for example, phenyl or naphthyl rings fused with one or more saturated or partially saturated carbon rings (e.g., bicyclo[4.2.0]octa-1,3,5-trienyl, or indanyl), which can be substituted at one or more carbon atoms of the aromatic and/or saturated or partially saturated rings.

As used herein, the term “aryl-(C₁-C₆)alkyl” refers to a functional group wherein a one-to-six carbon alkylene chain is attached to an aryl group, e.g., —CH₂CH₂-phenyl or —CH₂-phenyl (or benzyl). Specific examples are aryl-CH₂— and aryl-CH(CH₃)—. The term “substituted aryl-(C₁-C₆)alkyl” refers to an aryl-(C₁-C₆)alkyl functional group in which the aryl group is substituted. A specific example is substituted aryl(CH₂)—. Similarly, the term “heteroaryl-(C₁-C₆)alkyl” refers to a functional group wherein a one-to-three carbon alkylene chain is attached to a heteroaryl group, e.g., —CH₂CH₂-pyridyl. A specific example is heteroaryl-(CH₂)—. The term “substituted heteroaryl-(C₁-C₆)alkyl” refers to a heteroaryl-(C₁-C₆)alkyl functional group in which the heteroaryl group is substituted. A specific example is substituted heteroaryl-(CH₂)—.

As used herein, the term “cycloalkyl” by itself or as part of another substituent refers to, unless otherwise stated, a cyclic chain hydrocarbon having the number of carbon atoms designated (i.e., C₃-C₆ refers to a cyclic group comprising a ring group consisting of three to six carbon atoms) and includes straight, branched chain or cyclic substituent groups. Examples of (C₃-C₆)cycloalkyl groups are cyclopropyl, cyclobutyl, cyclopentyl and cyclohexyl. Cycloalkyl rings can be optionally substituted. Non-limiting examples of cycloalkyl groups include: cyclopropyl, 2-methyl-cyclopropyl, cyclopropenyl, cyclobutyl, 2,3-dihydroxycyclobutyl, cyclobutenyl, cyclopentyl, cyclopentenyl, cyclopentadienyl, cyclohexyl, cyclohexenyl, cycloheptyl, cyclooctanyl, decalinyl, 2,5-dimethylcyclopentyl, 3,5-dichlorocyclohexyl, 4-hydroxycyclohexyl, 3,3,5-trimethylcyclohex-1-yl, octahydropentalenyl, octahydro-1H-indenyl, 3a,4,5,6,7,7a-hexahydro-3H-inden-4-yl, decahydroazulenyl; bicyclo[6.2.0]decanyl, decahydronaphthalenyl, and dodecahydro-1H-fluorenyl. The term “cycloalkyl” also includes bicyclic hydrocarbon rings, non-limiting examples of which include, bicyclo[2.1.1]hexanyl, bicyclo[2.2.1]heptanyl, bicyclo[3.1.1]heptanyl, 1,3-dimethyl[2.2.1]heptan-2-yl, bicyclo[2.2.2]octanyl, and bicyclo[3.3.3]undecanyl.

As used herein, a “disease” is a state of health of a subject wherein the subject cannot maintain homeostasis, and wherein if the disease is not ameliorated then the subject's health continues to deteriorate.

As used herein, a “disorder” in a subject is a state of health in which the subject is able to maintain homeostasis, but in which the subject's state of health is less favorable than it would be in the absence of the disorder. Left untreated, a disorder does not necessarily cause a further decrease in the subject's state of health.

As used herein, the term “halide” refers to a halogen atom bearing a negative charge. The halide anions are fluoride (F⁻), chloride (Cl⁻), bromide (Br⁻), and iodide (I⁻).

As used herein, the term “halo” or “halogen” alone or as part of another substituent refers to, unless otherwise stated, a fluorine, chlorine, bromine, or iodine atom.

As used herein, the term “heteroalkenyl” by itself or in combination with another term refers to, unless otherwise stated, a stable straight or branched chain monounsaturated or diunsaturated hydrocarbon group consisting of the stated number of carbon atoms and one or two heteroatoms selected from the group consisting of O, N, and S, and wherein the nitrogen and sulfur atoms may optionally be oxidized and the nitrogen heteroatom may optionally be quaternized. Up to two heteroatoms may be placed consecutively. Examples include —CH—CH—O—CH₃, —CH═CH—CH₂—OH, —CH₂—CH═N—OCH₃, —CH—CH—N(CH₃)—CH₃, and —CH₂—CH═CH—CH₂—SH.

As used herein, the term “heteroalkyl” by itself or in combination with another term refers to, unless otherwise stated, a stable straight or branched chain alkyl group consisting of the stated number of carbon atoms and one or two heteroatoms selected from the group consisting of O, N, and S, and wherein the nitrogen and sulfur atoms may be optionally oxidized and the nitrogen heteroatom may be optionally quaternized. The heteroatom(s) may be placed at any position of the heteroalkyl group, including between the rest of the heteroalkyl group and the fragment to which it is attached, as well as attached to the most distal carbon atom in the heteroalkyl group. Examples include:—OCH₂CH₂CH₃, —CH₂CH₂CH₂OH, —CH₂CH₂NHCH₃, —CH₂SCH₂CH₃, and —CH₂CH₂S(═O)CH₃. Up to two heteroatoms may be consecutive, such as, for example, —CH₂NH—OCH₃, or —CH₂CH₂SSCH₃.

As used herein, the term “heteroaryl” or “heteroaromatic” refers to a heterocycle having aromatic character. A polycyclic heteroaryl may include one or more rings that are partially saturated. Examples include tetrahydroquinoline and 2,3-dihydrobenzofuryl.

As used herein, the term “heterocycle” or “heterocyclyl” or “heterocyclic” by itself or as part of another substituent refers to, unless otherwise stated, an unsubstituted or substituted, stable, mono- or multi-cyclic heterocyclic ring system that comprises carbon atoms and at least one heteroatom selected from the group consisting of N, O, and S, and wherein the nitrogen and sulfur heteroatoms may be optionally oxidized, and the nitrogen atom may be optionally quaternized. The heterocyclic system may be attached, unless otherwise stated, at any heteroatom or carbon atom that affords a stable structure. A heterocycle may be aromatic or non-aromatic in nature. In certain embodiments, the heterocycle is a heteroaryl.

Examples of non-aromatic heterocycles include monocyclic groups such as aziridine, oxirane, thiirane, azetidine, oxetane, thietane, pyrrolidine, pyrroline, imidazoline, pyrazolidine, dioxolane, sulfolane, 2,3-dihydrofuran, 2,5-dihydrofuran, tetrahydrofuran, thiophane, piperidine, 1,2,3,6-tetrahydropyridine, 1,4-dihydropyridine, piperazine, morpholine, thiomorpholine, pyran, 2,3-dihydropyran, tetrahydropyran, 1,4-dioxane, 1,3-dioxane, homopiperazine, homopiperidine, 1,3-dioxepane, 4,7-dihydro-1,3-dioxepin, and hexamethyleneoxide.

Examples of heteroaryl groups include pyridyl, pyrazinyl, pyrimidinyl (such as, but not limited to, 2- and 4-pyrimidinyl), pyridazinyl, thienyl, furyl, pyrrolyl, imidazolyl, thiazolyl, oxazolyl, pyrazolyl, isothiazolyl, 1,2,3-triazolyl, 1,2,4-triazolyl, 1,3,4-triazolyl, tetrazolyl, 1,2,3-thiadiazolyl, 1,2,3-oxadiazolyl, 1,3,4-thiadiazolyl, and 1,3,4-oxadiazolyl.

Examples of polycyclic heterocycles include indolyl (such as, but not limited to, 2-, 3-, 4-, 5-, 6- and 7-indolyl), indolinyl, quinolyl, tetrahydroquinolyl, isoquinolyl (such as, but not limited to, 1- and 5-isoquinolyl), 1,2,3,4-tetrahydroisoquinolyl, cinnolinyl, quinoxalinyl (such as, but not limited to, 2- and 5-quinoxalinyl), quinazolinyl, phthalazinyl, 1,8-naphthyridinyl, 1,4-benzodioxanyl, coumarin, dihydrocoumarin, 1,5-naphthyridinyl, benzofuryl (such as, but not limited to, 3-, 4-, 5-, 6- and 7-benzofuryl), 2,3-dihydrobenzofuryl, 1,2-benzisoxazolyl, benzothienyl (such as, but not limited to, 3-, 4-, 5-, 6-, and 7-benzothienyl), benzoxazolyl, benzothiazolyl (such as, but not limited to, 2-benzothiazolyl and 5-benzothiazolyl), purinyl, benzimidazolyl, benztriazolyl, thioxanthinyl, carbazolyl, carbolinyl, acridinyl, pyrrolizidinyl, and quinolizidinyl.

The aforementioned listing of heterocyclyl and heteroaryl moieties is intended to be representative and not limiting.

As used herein, the term “NMT” refers to Not More Than.

As used herein, the term “pharmaceutical composition” or “composition” refers to a mixture of at least one compound useful within the disclosure with a pharmaceutically acceptable carrier. The pharmaceutical composition facilitates administration of the compound to a subject.

As used herein, the term “pharmaceutically acceptable” refers to a material, such as a carrier or diluent, which does not abrogate the biological activity or properties of the compound useful within the disclosure, and is relatively non-toxic, i.e., the material may be administered to a subject without causing undesirable biological effects or interacting in a deleterious manner with any of the components of the composition in which it is contained.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the disclosure within or to the subject such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the disclosure, and not injurious to the subject. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound useful within the disclosure, and are physiologically acceptable to the subject. Supplementary active compounds may also be incorporated into the compositions. The “pharmaceutically acceptable carrier” may further include a pharmaceutically acceptable salt of the compound useful within the disclosure. Other additional ingredients that may be included in the pharmaceutical compositions used in the practice of the disclosure are known in the art and described, for example in Remington's Pharmaceutical Sciences (Genaro, Ed., Mack Publishing Co., 1985, Easton, PA), which is incorporated herein by reference.

As used herein, the language “pharmaceutically acceptable salt” refers to a salt of the administered compound prepared from pharmaceutically acceptable non-toxic acids and/or bases, including inorganic acids, inorganic bases, organic acids, inorganic bases, solvates (including hydrates) and clathrates thereof.

As used herein, a “pharmaceutically effective amount,” “therapeutically effective amount,” or “effective amount” of a compound is that amount of compound that is sufficient to provide a beneficial effect to the subject to which the compound is administered.

The term “prevent,” “preventing,” or “prevention” as used herein means avoiding or delaying the onset of symptoms associated with a disease or condition in a subject that has not developed such symptoms at the time the administering of an agent or compound commences. Disease, condition and disorder are used interchangeably herein.

By the term “specifically bind” or “specifically binds” as used herein is meant that a first molecule preferentially binds to a second molecule (e.g., a particular receptor or enzyme), but does not necessarily bind only to that second molecule.

As used herein, the terms “subject” and “individual” and “patient” can be used interchangeably and may refer to a human or non-human mammal or a bird. Non-human mammals include, for example, livestock and pets, such as ovine, bovine, porcine, canine, feline and murine mammals. In certain embodiments, the subject is human.

As used herein, the term “substituted” refers to that an atom or group of atoms has replaced hydrogen as the substituent attached to another group.

As used herein, the term “substituted alkyl,” “substituted cycloalkyl,” “substituted alkenyl,” or “substituted alkynyl” refers to alkyl, cycloalkyl, alkenyl, or alkynyl, as defined elsewhere herein, substituted by one, two or three substituents independently selected from the group consisting of halogen, —OH, alkoxy, tetrahydro-2-H-pyranyl, —NH₂, —NH(C₁-C₆ alkyl), —N(C₁-C₆ alkyl)₂, 1-methyl-imidazol-2-yl, pyridin-2-yl, pyridin-3-yl, pyridin-4-yl, —C(═O)OH, —C(═O)O(C₁-C₆)alkyl, trifluoromethyl, —C≡N, —C(═O)NH₂, —C(═O)NH(C₁-C₆)alkyl, —C(═O)N((C₁-C₆)alkyl)₂, —SO₂NH₂, —SO₂NH(C₁-C₆ alkyl), —SO₂N(C₁-C₆ alkyl)₂, —C(═NH)NH₂, and —NO₂, in certain embodiments containing one or two substituents independently selected from halogen, —OH, alkoxy, —NH₂, trifluoromethyl, —N(CH₃)₂, and —C(═O)OH, in certain embodiments independently selected from halogen, alkoxy and —OH. Examples of substituted alkyls include, but are not limited to, 2,2-difluoropropyl, 2-carboxycyclopentyl and 3-chloropropyl.

For aryl, aryl-(C₁-C₃)alkyl and heterocyclyl groups, the term “substituted” as applied to the rings of these groups refers to any level of substitution, namely mono-, di-, tri-, tetra-, or penta-substitution, where such substitution is permitted. The substituents are independently selected, and substitution may be at any chemically accessible position. In certain embodiments, the substituents vary in number between one and four. In other embodiments, the substituents vary in number between one and three. In yet another embodiments, the substituents vary in number between one and two. In yet other embodiments, the substituents are independently selected from the group consisting of C₁-C₆ alkyl, —OH, C₁-C₆ alkoxy, halogen, amino, acetamido and nitro. As used herein, where a substituent is an alkyl or alkoxy group, the carbon chain may be branched, straight or cyclic.

Unless otherwise noted, when two substituents are taken together to form a ring having a specified number of ring atoms (e.g., R² and R³ taken together with the nitrogen to which they are attached to form a ring having from 3 to 7 ring members), the ring can have carbon atoms and optionally one or more (e.g., 1 to 3) additional heteroatoms independently selected from nitrogen, oxygen, or sulfur. The ring can be saturated or partially saturated, and can be optionally substituted.

Whenever a term or either of their prefix roots appear in a name of a substituent the name is to be interpreted as including those limitations provided herein. For example, whenever the term “alkyl” or “aryl” or either of their prefix roots appear in a name of a substituent (e.g., arylalkyl, alkylamino) the name is to be interpreted as including those limitations given elsewhere herein for “alkyl” and “aryl” respectively.

In certain embodiments, substituents of compounds are disclosed in groups or in ranges. It is specifically intended that the description include each and every individual subcombination of the members of such groups and ranges. For example, the term “C₁₆ alkyl” is specifically intended to individually disclose C₁, C₂, C₃, C₄, C₅, C₆, C₁-C₆, C₁-C₅, C₁-C₄, C₁-C₃, C₁-C₂, C₂-C₆, C₂-C₅, C₂-C₄, C₂-C₃, C₃-C₆, C₃-C₅, C₃-C₄, C₄-C₆, C₄-C₅, and C₅-C₆ alkyl.

The terms “treat,” “treating” and “treatment,” as used herein, means reducing the frequency or severity with which symptoms of a disease or condition are experienced by a subject by virtue of administering an agent or compound to the subject.

Certain abbreviations used herein follow: cccDNA, covalently closed circular DNA; DMSO, dimethylsulfoxide; HBsAg, HBV surface antigen; HBV, hepatitis B virus; HDV, hepatitis D virus; HPLC, high pressure liquid chromatography; LCMS, liquid chromatography mass spectrometry; NARTI or NRTI, reverse-transcriptase inhibitor; NMR, Nuclear Magnetic Resonance; NtARTI or NtRTI, nucleotide analog reverse-transcriptase inhibitor; pg RNA, pregenomic RNA; rcDNA, relaxed circular DNA; RT, retention time; sAg, surface antigen; TLC, thin layer chromatography.

Ranges: throughout this disclosure, various aspects of the present disclosure can be presented in a range format. It should be understood that the description in range format is merely for convenience and brevity and should not be construed as an inflexible limitation on the scope of the present disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise. This applies regardless of the breadth of the range.

### Synthesis

The present disclosure further provides methods of preparing compounds of the present disclosure. Compounds of the present teachings can be prepared in accordance with the procedures outlined herein, from commercially available starting materials, compounds known in the literature, or readily prepared intermediates, by employing standard synthetic methods and procedures known to those skilled in the art. Standard synthetic methods and procedures for the preparation of organic molecules and functional group transformations and manipulations can be readily obtained from the relevant scientific literature or from standard textbooks in the field.

It is appreciated that where typical or preferred process conditions (i.e., reaction temperatures, times, mole ratios of reactants, solvents, pressures, and so forth) are given, other process conditions can also be used unless otherwise stated. Optimum reaction conditions can vary with the particular reactants or solvent used, but such conditions can be determined by one skilled in the art by routine optimization procedures. Those skilled in the art of organic synthesis will recognize that the nature and order of the synthetic steps presented can be varied for the purpose of optimizing the formation of the compounds described herein.

The processes described herein can be monitored according to any suitable method known in the art. For example, product formation can be monitored by spectroscopic means, such as nuclear magnetic resonance spectroscopy (e.g., ¹H or ¹³C), infrared spectroscopy, spectrophotometry (e.g., UV-visible), mass spectrometry, or by chromatography such as high-performance liquid chromatograpy (HPLC), gas chromatography (GC), gel-permeation chromatography (GPC), or thin layer chromatography (TLC).

Preparation of the compounds can involve protection and deprotection of various chemical groups. The need for protection and deprotection and the selection of appropriate protecting groups can be readily determined by one skilled in the art. The chemistry of protecting groups can be found, for example, in Greene, et al., Protective Groups in Organic Synthesis, 2d. Ed. (Wiley & Sons, 1991), the entire disclosure of which is incorporated by reference herein for all purposes.

The reactions or the processes described herein can be carried out in suitable solvents that can be readily selected by one skilled in the art of organic synthesis. Suitable solvents typically are substantially nonreactive with the reactants, intermediates, and/or products at the temperatures at which the reactions are carried out, i.e., temperatures that can range from the solvent's freezing temperature to the solvent's boiling temperature. A given reaction can be carried out in one solvent or a mixture of more than one solvent. Depending on the particular reaction step, suitable solvents for a particular reaction step can be selected.

The disclosure includes methods of preparing (S)—N-(8,9-difluoro-6-oxo-1,4,5,6-tetrahydro-2H-pyrano[3,4-c]isoquinolin-1-yl)-5,6-difluoro-N-methyl-1H-indole-2-carboxamide, also known as compound (X), or a salt, solvate, prodrug, isotopically labelled derivative, stereoisomer (such as, in a non-limiting example, an enantiomer or any mixtures thereof, such as, in a non-limiting example, mixtures in any proportions of enantiomers thereof), and/or tautomer, and any mixtures thereof:

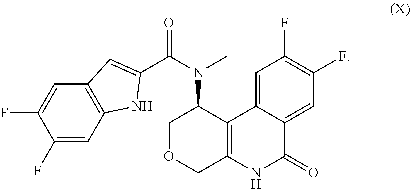 [SMILES: CN(C(=O)c1cc2cc(F)c(F)cc2[nH]1)[C@@H]1COCc2[nH]c(=O)c3cc(F)c(F)cc3c21]

In certain embodiments, the compound of formula (X), or a salt, solvate, prodrug, isotopically labelled derivative, and/or tautomer thereof, can be prepared according to the non-limiting synthetic scheme outlined in Schemes 1-2.

Commercially available aryl bromide (A) can be converted to the corresponding isochromenone (B), for example in a two-step process, wherein (A) is reacted with pyran-3,5-dione in the presence of a transition metal catalyst, such as but not limited to a copper (I) salt, in the presence of a base, such as but not limited to an alkali carbonate, to generate a keto-acid intermediate, which can undergo acid-catalyzed cyclization to yield isochromenone (B).

Isochromenone (B) can be reacted with an ammonium salt, such as but not limited to ammonium hydroxide, to provide the corresponding isoquinolinone (C).

Isoquinolinone (C) can be converted to benzylamine (D), for example, in a two-step process, wherein (C) is reacted with an α-methyl benzylamine in the presence of a Lewis acid, such as but not limited to a titanium (IV) alkoxide, to generate an imine intermediate, which can be reduced to benzylamine (D) using a reducing agent such as but not limited to a borohydride reagent, such as but not limited to NaBH₄.

Benzylamine (D) can be methylated at the benzylic nitrogen by reductive alkylation, using formaldehyde, or a formaldehyde donor such as but not limited to paraformaldehyde, in the presence of an acid to generate an iminium intermediate, which can be reduced to tertiary amine (E) using a reducing agent, such as but not limited to NaBH(OAc)₃ (i.e., STAB).

Tertiary amine (E) can be reductively cleaved to the corresponding methyl amine (F) using a suitable reducing agent, such as but not limited to H₂ in the presence of Pd/C.

Acid chloride (H) can be prepared by reacting carboxylic acid (G) with a suitable acyl chlorinating reagent, including but not limited to oxalyl chloride and thionyl chloride.

Compound (F) and compound (H) can be coupled in the presence of a base, including but not limited to an alkali carbonate, to provide compound (X).

In certain embodiments, isoquinolinone (C) can be converted to benzylamine (D′), for example, in a two-step process, wherein (C) is reacted with an α-methyl 4-methoxy-benzylamine in the presence of a Lewis acid, such as but not limited to a titanium (IV) alkoxide, to generate an imine intermediate, which can be reduced to benzylamine (D′) using a reducing agent such as but not limited to a borohydride, such as but not limited to NaBH₄.

4-Methoxy-benzylamine (D′) can be methylated at the benzylic nitrogen by reductive alkylation, using formaldehyde, or a formaldehyde donor such as but not limited to paraformaldehyde, in the presence of an acid to generate an iminium intermediate, which can be reduced to tertiary amine (E′) using a reducing agent, such as but not limited to NaBH(OAc)₃ (i.e., STAB).

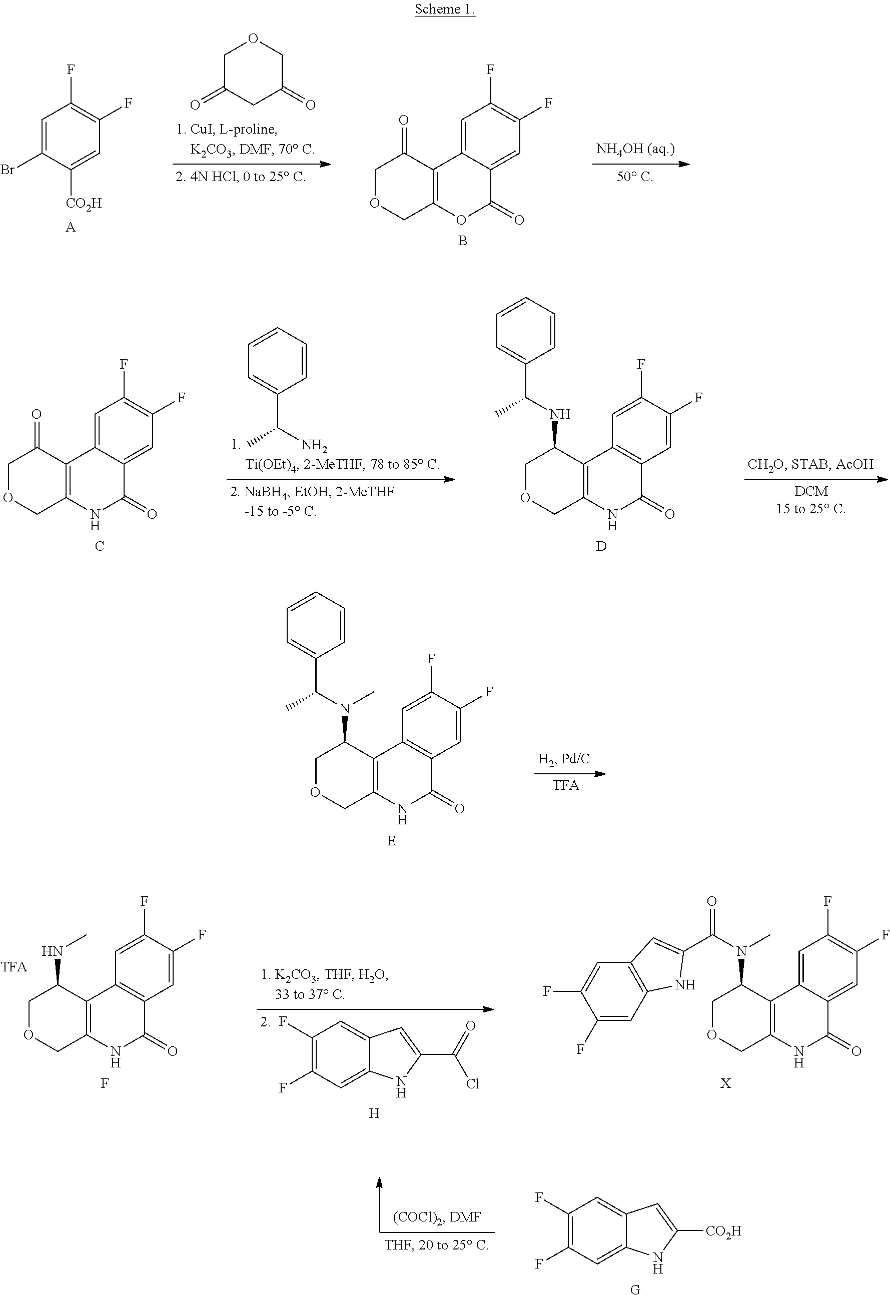

Tertiary amine (E′) can be cleaved to the corresponding methyl amine (F) using an acid, such as but not limited to trifluoroacetic acid (TFA). In certain embodiments, cleavage of (E′) to (F) further comprises a trialkylsilane, such as but not limited to triethylsilane.

In certain embodiments, compound (X) can be prepared from compound (F) and compound (H), as described elsewhere herein.

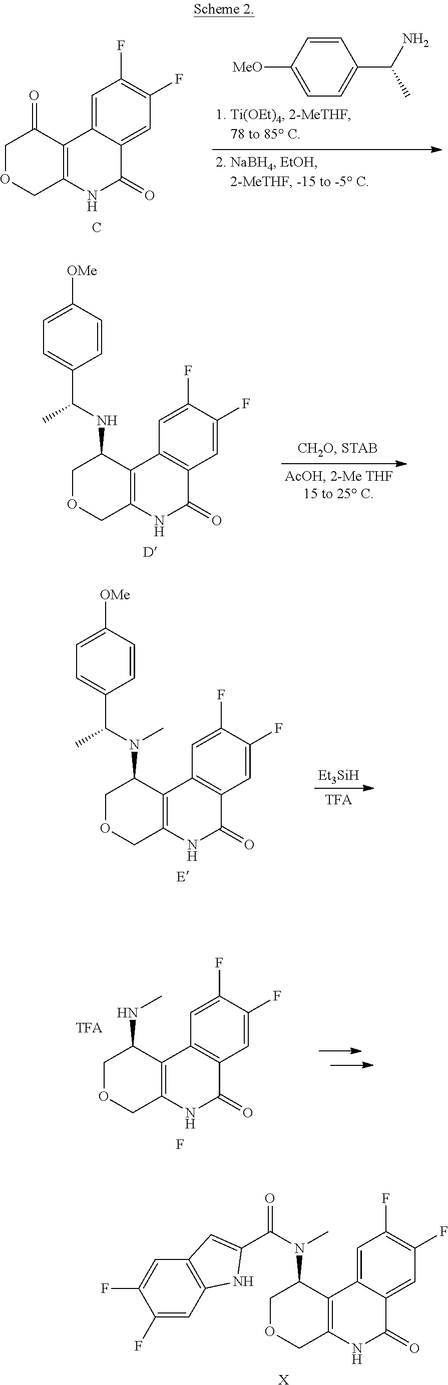

The compounds of the disclosure may possess one or more stereocenters, and each stereocenter may exist independently in either the (R)- or (S)-configuration. In certain embodiments, compounds described herein are present in optically active or racemic forms. The compounds described herein encompass racemic, optically active, regioisomeric and stereoisomeric forms, or combinations thereof that possess the therapeutically useful properties described herein. Preparation of optically active forms is achieved in any suitable manner, including, by way of non-limiting example, by resolution of the racemic form with recrystallization techniques, synthesis from optically active starting materials, chiral synthesis, or chromatographic separation using a chiral stationary phase. A compound illustrated herein by the racemic formula further represents either of the two enantiomers or any mixtures thereof, or in the case where two or more chiral centers are present, all diastereomers or any mixtures thereof.

In certain embodiments, the compounds of the disclosure exist as tautomers. All tautomers are included within the scope of the compounds recited herein.

Compounds described herein also include isotopically labeled compounds wherein one or more atoms is replaced by an atom having the same atomic number, but an atomic mass or mass number different from the atomic mass or mass number usually found in nature. Examples of isotopes suitable for inclusion in the compounds described herein include and are not limited to ²H, ³H, ¹¹C, ¹³C, ¹⁴C, ³⁶Cl, ¹⁸F, ¹²³I, ¹²⁵I, ¹³N, ¹⁵N, ¹⁵O, ¹⁷O, ¹⁸O, ³²P, and ³⁵S. In certain embodiments, substitution with heavier isotopes such as deuterium affords greater chemical stability. Isotopically labeled compounds are prepared by any suitable method or by processes using an appropriate isotopically labeled reagent in place of the non-labeled reagent otherwise employed.

In certain embodiments, the compounds described herein are labeled by other means, including, but not limited to, the use of chromophores or fluorescent moieties, bioluminescent labels, or chemiluminescent labels.

In all of the embodiments provided herein, examples of suitable optional substituents are not intended to limit the scope of the claimed disclosure. The compounds of the disclosure may contain any of the substituents, or combinations of substituents, provided herein.

### Salts

The compounds described herein may form salts with acids or bases, and such salts are included in the present disclosure. The term “salts” embraces addition salts of free acids or bases that are useful within the methods of the disclosure. The term “pharmaceutically acceptable salt” refers to salts that possess toxicity profiles within a range that affords utility in pharmaceutical applications. In certain embodiments, the salts are pharmaceutically acceptable salts. Pharmaceutically unacceptable salts may nonetheless possess properties such as high crystallinity, which have utility in the practice of the present disclosure, such as for example utility in process of synthesis, purification or formulation of compounds useful within the methods of the disclosure.

Suitable pharmaceutically acceptable acid addition salts may be prepared from an inorganic acid or from an organic acid. Examples of inorganic acids include sulfate, hydrogen sulfate, hydrochloric, hydrobromic, hydriodic, nitric, carbonic, sulfuric, and phosphoric acids (including hydrogen phosphate and dihydrogen phosphate). Appropriate organic acids may be selected from aliphatic, cycloaliphatic, aromatic, araliphatic, heterocyclic, carboxylic and sulfonic classes of organic acids, examples of which include formic, acetic, propionic, succinic, glycolic, gluconic, lactic, malic, tartaric, citric, ascorbic, glucuronic, maleic, fumaric, pyruvic, aspartic, glutamic, benzoic, anthranilic, 4-hydroxybenzoic, phenylacetic, mandelic, embonic (or pamoic), methanesulfonic, ethanesulfonic, benzenesulfonic, pantothenic, sulfanilic, 2-hydroxyethanesulfonic, trifluoromethanesulfonic, p-toluenesulfonic, cyclohexylaminosulfonic, stearic, alginic, β-hydroxybutyric, salicylic, galactaric, galacturonic acid, glycerophosphonic acids and saccharin (e.g., saccharinate, saccharate). Salts may be comprised of a fraction of one, one or more than one molar equivalent of acid or base with respect to any compound of the disclosure.

Suitable pharmaceutically acceptable base addition salts of compounds of the disclosure include, for example, ammonium salts and metallic salts including alkali metal, alkaline earth metal and transition metal salts such as, for example, calcium, magnesium, potassium, sodium and zinc salts. Pharmaceutically acceptable base addition salts also include organic salts made from basic amines such as, for example, N,N′-dibenzylethylene-diamine, chloroprocaine, choline, diethanolamine, ethylenediamine, meglumine (or N-methylglucamine) and procaine. All of these salts may be prepared from the corresponding compound by reacting, for example, the appropriate acid or base with the compound.

### Combination Therapies

In one aspect, the compounds of the disclosure are useful within the methods of the disclosure in combination with one or more additional agents useful for treating HBV and/or HDV infections. These additional agents may comprise compounds or compositions identified herein, or compounds (e.g., commercially available compounds) known to treat, prevent, or reduce the symptoms of HBV and/or HDV infections.

Non-limiting examples of one or more additional agents useful for treating HBV and/or HDV infections include: (a) reverse transcriptase inhibitors; (b) capsid inhibitors; (c) cccDNA formation inhibitors; (d) RNA destabilizers; (e) oligomeric nucleotides targeted against the HBV genome; (f) immunostimulators, such as checkpoint inhibitors (e.g., PD-L1 inhibitors); (g) GalNAc-siRNA conjugates targeted against an HBV gene transcript; and (h) therapeutic vaccine.

### (a) Reverse Transcriptase Inhibitors

In certain embodiments, the reverse transcriptase inhibitor is a reverse-transcriptase inhibitor (NARTI or NRTI). In other embodiments, the reverse transcriptase inhibitor is a nucleotide analog reverse-transcriptase inhibitor (NtARTI or NtRTI).

Reported reverse transcriptase inhibitors include, but are not limited to, entecavir, clevudine, telbivudine, lamivudine, adefovir, and tenofovir, tenofovir disoproxil, tenofovir alafenamide, adefovir dipovoxil, (1R,2R,3R,5R)-3-(6-amino-9H-9-purinyl)-2-fluoro-5-(hydroxymethyl)-4-methylenecyclopentan-1-ol (described in U.S. Pat. No. 8,816,074, incorporated herein in its entirety by reference), emtricitabine, abacavir, elvucitabine, ganciclovir, lobucavir, famciclovir, penciclovir, and amdoxovir.

Reported reverse transcriptase inhibitors further include, but are not limited to, entecavir, lamivudine, and (1R,2R,3R,5R)-3-(6-amino-9H-9-purinyl)-2-fluoro-5-(hydroxymethyl)-4-methylenecyclopentan-1-ol.

Reported reverse transcriptase inhibitors further include, but are not limited to, a covalently bound phosphoramidate or phosphonamidate moiety of the above-mentioned reverse transcriptase inhibitors, or as described in for example U.S. Pat. No. 8,816,074, US Patent Application Publications No. US 2011/0245484 A1, and US 2008/0286230A1, all of which incorporated herein in their entireties by reference.

Reported reverse transcriptase inhibitors further include, but are not limited to, nucleotide analogs that comprise a phosphoramidate moiety, such as, for example, methyl ((((1R,3R,4R,5R)-3-(6-amino-9H-purin-9-yl)-4-fluoro-5-hydroxy-2-methylenecyclopentyl) methoxy)(phenoxy) phosphoryl)-(D or L)-alaninate and methyl ((((1R,2R,3R,4R)-3-fluoro-2-hydroxy-5-methylene-4-(6-oxo-1,6-dihydro-9H-purin-9-yl)cyclopentyl)methoxy)(phenoxy) phosphoryl)-(D or L)-alaninate. Also included are the individual diastereomers thereof, which include, for example, methyl ((R)-(((1R,3R,4R,5R)-3-(6-amino-9H-purin-9-yl)-4-fluoro-5-hydroxy-2-methylenecyclopentyl)methoxy)(phenoxy)phosphoryl)-(D or L)-alaninate and methyl ((S)-(((1R,3R,4R,5R)-3-(6-amino-9H-purin-9-yl)-4-fluoro-5-hydroxy-2-methylenecyclopentyl) methoxy)(phenoxy)phosphoryl)-(D or L)-alaninate.

Reported reverse transcriptase inhibitors further include, but are not limited to, compounds comprising a phosphonamidate moiety, such as, for example, tenofovir alafenamide, as well as those described in U.S. Patent Application Publication No. US 2008/0286230 A1, incorporated herein in its entirety by reference. Methods for preparing stereoselective phosphoramidate or phosphonamidate containing actives are described in, for example, U.S. Pat. No. 8,816,074, as well as U.S. Patent Application Publications No. US 2011/0245484 A1 and US 2008/0286230 A1, all of which incorporated herein in their entireties by reference.

### (b) Capsid Inhibitors

As described herein, the term “capsid inhibitor” includes compounds that are capable of inhibiting the expression and/or function of a capsid protein either directly or indirectly. For example, a capsid inhibitor may include, but is not limited to, any compound that inhibits capsid assembly, induces formation of non-capsid polymers, promotes excess capsid assembly or misdirected capsid assembly, affects capsid stabilization, and/or inhibits encapsidation of RNA (pgRNA). Capsid inhibitors also include any compound that inhibits capsid function in a downstream event(s) within the replication process (e.g., viral DNA synthesis, transport of relaxed circular DNA (rcDNA) into the nucleus, covalently closed circular DNA (cccDNA) formation, virus maturation, budding and/or release, and the like). For example, in certain embodiments, the inhibitor detectably inhibits the expression level or biological activity of the capsid protein as measured, e.g., using an assay described herein. In certain embodiments, the inhibitor inhibits the level of rcDNA and downstream products of viral life cycle by at least 5%, at least 10%, at least 20%, at least 50%, at least 75%, or at least 90%.

Reported capsid inhibitors include, but are not limited to, compounds described in International Patent Applications Publication Nos WO 2013006394, WO 2014106019, and WO2014089296, all of which incorporated herein in their entireties by reference.

Reported capsid inhibitors also include, but are not limited to, the following compounds and pharmaceutically acceptable salts and/or solvates thereof: Bay-41-4109 (see Int'l Patent Application Publication No. WO 2013144129), AT-61 (see Int'l Patent Application Publication No. WO 1998033501; and King, et al., 1998, Antimicrob. Agents Chemother. 42(12):3179-3186), DVR-01 and DVR-23 (see Int'l Patent Application Publication No. WO 2013006394; and Campagna, et al., 2013, J. Virol. 87(12):6931, all of which incorporated herein in their entireties by reference.

In addition, reported capsid inhibitors include, but are not limited to, those generally and specifically described in U.S. Patent Application Publication Nos. US 2015/0225355, US 2015/0132258, US 2016/0083383, US 2016/0052921 and Int'l Patent Application Publication Nos. WO 2013096744, WO 2014165128, WO 2014033170, WO 2014033167, WO 2014033176, WO 2014131847, WO 2014161888, WO 2014184350, WO 2014184365, WO 2015059212, WO 2015011281, WO 2015118057, WO 2015109130, WO 2015073774, WO 2015180631, WO 2015138895, WO 2016089990, WO 2017015451, WO 2016183266, WO 2017011552, WO 2017048950, WO 2017048954, WO 2017048962, WO 2017064156 and are incorporated herein in their entirety by reference.

(c) cccDNA Formation Inhibitors

Covalently closed circular DNA (cccDNA) is generated in the cell nucleus from viral rcDNA and serves as the transcription template for viral mRNAs. As described herein, the term “cccDNA formation inhibitor” includes compounds that are capable of inhibiting the formation and/or stability of cccDNA either directly or indirectly. For example, a cccDNA formation inhibitor may include, but is not limited to, any compound that inhibits capsid disassembly, rcDNA entry into the nucleus, and/or the conversion of reDNA into cccDNA. For example, in certain embodiments, the inhibitor detectably inhibits the formation and/or stability of the cccDNA as measured, e.g., using an assay described herein. In certain embodiments, the inhibitor inhibits the formation and/or stability of cccDNA by at least 5%, at least 10%, at least 20%, at least 50%, at least 75%, or at least 90%.

Reported cccDNA formation inhibitors include, but are not limited to, compounds described in Int'l Patent Application Publication No. WO 2013130703, and are incorporated herein in their entirety by reference.

In addition, reported cccDNA formation inhibitors include, but are not limited to, those generally and specifically described in U.S. Patent Application Publication No. US 2015/0038515 A1, and are incorporated herein in their entirety by reference.

### (d) RNA Destabilizer

As used herein, the term “RNA destabilizer” refers to a molecule, or a salt or solvate thereof, that reduces the total amount of HBV RNA in mammalian cell culture or in a live human subject. In a non-limiting example, an RNA destabilizer reduces the amount of the RNA transcript(s) encoding one or more of the following HBV proteins: surface antigen, core protein, RNA polymerase, and e antigen. In certain embodiments, the RNA destabilizer reduces the total amount of HBV RNA in mammalian cell culture or in a live human subject by at least 5%, at least 10%, at least 20%, at least 50%, at least 75%, or at least 90%.

Reported RNA destabilizers include compounds described in U.S. Pat. No. 8,921,381, as well as compounds described in U.S. Patent Application Publication Nos. US 2015/0087659 and US 2013/0303552, all of which are incorporated herein in their entireties by reference.

In addition, reported RNA destabilizers include, but are not limited to, those generally and specifically described in Int'l Patent Application Publication Nos. WO 2015113990, WO 2015173164, US 2016/0122344, WO 2016107832, WO 2016023877, WO 2016128335, WO 2016177655, WO 2016071215, WO 2017013046, WO 2017016921, WO 2017016960, WO 2017017042, WO 2017017043, WO 2017102648, WO 2017108630, WO 2017114812, WO 2017140821, WO 2018085619, and are incorporated herein in their entirety by reference.

### (e) Oligomeric Nucleotides Targeted Against the HBV Genome

Reported oligomeric nucleotides targeted against the HBV genome include, but are not limited to, Arrowhead-ARC-520 (see U.S. Pat. No. 8,809,293; and Wooddell et al., 2013, Molecular Therapy 21(5):973-985, all of which incorporated herein in their entireties by reference).

In certain embodiments, the oligomeric nucleotides can be designed to target one or more genes and/or transcripts of the HBV genome. Oligomeric nucleotide targeted to the HBV genome also include, but are not limited to, isolated, double stranded, siRNA molecules, that each include a sense strand and an antisense strand that is hybridized to the sense strand. In certain embodiments, the siRNA target one or more genes and/or transcripts of the HBV genome.

### (f) Immunostimulators

### Checkpoint Inhibitors

As described herein, the term “checkpoint inhibitor” includes any compound that is capable of inhibiting immune checkpoint molecules that are regulators of the immune system (e.g., stimulate or inhibit immune system activity). For example, some checkpoint inhibitors block inhibitory checkpoint molecules, thereby stimulating immune system function, such as stimulation of T cell activity against cancer cells. A non-limiting example of a checkpoint inhibitor is a PD-L1 inhibitor.

As described herein, the term “PD-L1 inhibitor” includes any compound that is capable of inhibiting the expression and/or function of the protein Programmed Death-Ligand 1 (PD-L1) either directly or indirectly. PD-L1, also known as cluster of differentiation 274 (CD274) or B7 homolog 1 (B7-H1), is a type 1 transmembrane protein that plays a major role in suppressing the adaptive arm of immune system during pregnancy, tissue allograft transplants, autoimmune disease, and hepatitis. PD-L1 binds to its receptor, the inhibitory checkpoint molecule PD-1 (which is found on activated T cells, B cells, and myeloid cells) so as to modulate activation or inhibition of the adaptive arm of immune system. In certain embodiments, the PD-L1 inhibitor inhibits the expression and/or function of PD-L1 by at least 5%, at least 10%, at least 20%, at least 50%, at least 75%, or at least 90%.

Reported PD-L1 Inhibitors include, but are not limited to, compounds recited in one of the following patent application publications: US 2018/0057455; US 2018/0057486; WO 2017/106634; WO 2018/026971; WO 2018/045142; WO 2018/118848; WO 2018/119221; WO 2018/119236; WO 2018/119266; WO 2018/119286; WO 2018/121560; WO 2019/076343; WO 2019/087214; and are incorporated herein in their entirety by reference.

### (g) GalNAc-siRNA Conjugates Targeted Against an HBV Gene Transcript

“GalNAc” is the abbreviation for N-acetylgalactosamine, and “siRNA” is the abbreviation for small interfering RNA. An siRNA that targets an HBV gene transcript is covalently bonded to GalNAc in a GalNAc-siRNA conjugate useful in the practice of the present disclosure. While not wishing to be bound by theory, it is believed that GalNAc binds to asialoglycoprotein receptors on hepatocytes thereby facilitating the targeting of the siRNA to the hepatocytes that are infected with HBV. The siRNA enter the infected hepatocytes and stimulate destruction of HBV gene transcripts by the phenomenon of RNA interference.

Examples of GalNAc-siRNA conjugates useful in the practice of this aspect of the present disclosure are set forth in published international application PCT/CA2017/050447 (PCT Application Publication number WO/2017/177326, published on Oct. 19, 2017) which is hereby incorporated by reference in its entirety.

### (h) Therapeutic Vaccines

In certain embodiments, administration of a therapeutic vaccine is useful in the practice of the present disclosure for the treatment of a viral disease in a subject. In certain embodiments, the viral disease is a hepatitis virus. In certain embodiments, the hepatitis virus is at least one selected from the group consisting of hepatitis B virus (HBV) and hepatitis D virus (HDV). In certain embodiments, the subject is a human.

A synergistic effect may be calculated, for example, using suitable methods such as, for example, the Sigmoid-Emax equation (Holford & Scheiner, 1981, Clin. Pharmacokinet. 6:429-453), the equation of Loewe additivity (Loewe & Muischnek, 1926, Arch. Exp. Pathol Pharmacol. 114: 313-326) and the median-effect equation (Chou & Talalay, 1984, Adv. Enzyme Regul. 22:27-55). Each equation referred to elsewhere herein may be applied to experimental data to generate a corresponding graph to aid in assessing the effects of the drug combination. The corresponding graphs associated with the equations referred to elsewhere herein are the concentration-effect curve, isobologram curve and combination index curve, respectively.

### Methods

The current disclosure, in one aspect, provides a method of treating or preventing hepatitis virus infection in a subject. In certain embodiments, the infection comprises hepatitis B virus (HBV) infection. In other embodiments, the method comprises administering to the subject in need thereof a therapeutically effective amount of at least one compound of the disclosure. In yet other embodiments, the at least one compound is administered to the subject in a pharmaceutically acceptable composition. In yet other embodiments, the subject is further administered at least one additional agent useful for treating the hepatitis infection. In yet other embodiments, the at least one additional agent comprises at least one selected from the group consisting of reverse transcriptase inhibitor; capsid inhibitor; cccDNA formation inhibitor; RNA destabilizer; oligomeric nucleotide targeted against the HBV genome; immunostimulator, such as checkpoint inhibitor (e.g., PD-L1 inhibitor); GalNAc-siRNA conjugate targeted against an HBV gene transcript; and therapeutic vaccine. In yet other embodiments, the subject is co-administered the at least one compound and the at least one additional agent. In yet other embodiments, the at least one compound and the at least one additional agent are coformulated.

The disclosure further provides a method of inhibiting expression and/or function of a viral capsid protein either directly or indirectly in a subject. In certain embodiments, the method comprises administering to the subject in need thereof a therapeutically effective amount of at least one compound of the disclosure. In other embodiments, the at least one compound is administered to the subject in a pharmaceutically acceptable composition. In yet other embodiments, the subject is further administered at least one additional agent useful for treating HBV infection. In yet other embodiments, the at least one additional agent comprises at least one selected from the group consisting of reverse transcriptase inhibitor; capsid inhibitor; cccDNA formation inhibitor; RNA destabilizer; oligomeric nucleotide targeted against the HBV genome; immunostimulator, such as checkpoint inhibitor (e.g., PD-L1 inhibitor); GalNAc-siRNA conjugate targeted against an HBV gene transcript; and therapeutic vaccine. In yet other embodiments, the subject is co-administered the at least one compound and the at least one additional agent. In yet other embodiments, the at least one compound and the at least one additional agent are coformulated.

In certain embodiments, the subject is a mammal. In other embodiments, the mammal is a human.

### Pharmaceutical Compositions and Formulations

The disclosure provides pharmaceutical compositions comprising at least one compound of the disclosure or a salt or solvate thereof, which are useful to practice methods of the disclosure. Such a pharmaceutical composition may consist of at least one compound of the disclosure or a salt or solvate thereof, in a form suitable for administration to a subject, or the pharmaceutical composition may comprise at least one compound of the disclosure or a salt or solvate thereof, and one or more pharmaceutically acceptable carriers, one or more additional ingredients, or some combination of these. At least one compound of the disclosure may be present in the pharmaceutical composition in the form of a physiologically acceptable salt, such as in combination with a physiologically acceptable cation or anion, as is well known in the art.

In certain embodiments, the pharmaceutical compositions useful for practicing the method of the disclosure may be administered to deliver a dose of between 1 ng/kg/day and 100 mg/kg/day. In other embodiments, the pharmaceutical compositions useful for practicing the disclosure may be administered to deliver a dose of between 1 ng/kg/day and 1,000 mg/kg/day.

The relative amounts of the active ingredient, the pharmaceutically acceptable carrier, and any additional ingredients in a pharmaceutical composition of the disclosure will vary, depending upon the identity, size, and condition of the subject treated and further depending upon the route by which the composition is to be administered. By way of example, the composition may comprise between 0.1% and 100% (w/w) active ingredient.

Pharmaceutical compositions that are useful in the methods of the disclosure may be suitably developed for nasal, inhalational, oral, rectal, vaginal, pleural, peritoneal, parenteral, topical, transdermal, pulmonary, intranasal, buccal, ophthalmic, epidural, intrathecal, intravenous or another route of administration. A composition useful within the methods of the disclosure may be directly administered to the brain, the brainstem, or any other part of the central nervous system of a mammal or bird. Other contemplated formulations include projected nanoparticles, microspheres, liposomal preparations, coated particles, polymer conjugates, resealed erythrocytes containing the active ingredient, and immunologically-based formulations.

In certain embodiments, the compositions of the disclosure are part of a pharmaceutical matrix, which allows for manipulation of insoluble materials and improvement of the bioavailability thereof, development of controlled or sustained release products, and generation of homogeneous compositions. By way of example, a pharmaceutical matrix may be prepared using hot melt extrusion, solid solutions, solid dispersions, size reduction technologies, molecular complexes (e.g., cyclodextrins, and others), microparticulate, and particle and formulation coating processes. Amorphous or crystalline phases may be used in such processes.

The route(s) of administration will be readily apparent to the skilled artisan and will depend upon any number of factors including the type and severity of the disease being treated, the type and age of the veterinary or human patient being treated, and the like.

The formulations of the pharmaceutical compositions described herein may be prepared by any method known or hereafter developed in the art of pharmacology and pharmaceutics. In general, such preparatory methods include the step of bringing the active ingredient into association with a carrier or one or more other accessory ingredients, and then, if necessary or desirable, shaping or packaging the product into a desired single-dose or multi-dose unit.

As used herein, a “unit dose” is a discrete amount of the pharmaceutical composition comprising a predetermined amount of the active ingredient. The amount of the active ingredient is generally equal to the dosage of the active ingredient that would be administered to a subject or a convenient fraction of such a dosage such as, for example, one-half or one-third of such a dosage. The unit dosage form may be for a single daily dose or one of multiple daily doses (e.g., about 1 to 4 or more times per day). When multiple daily doses are used, the unit dosage form may be the same or different for each dose.

Although the descriptions of pharmaceutical compositions provided herein are principally directed to pharmaceutical compositions suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the disclosure is contemplated include, but are not limited to, humans and other primates, mammals including commercially relevant mammals such as cattle, pigs, horses, sheep, cats, and dogs.

In certain embodiments, the compositions of the disclosure are formulated using one or more pharmaceutically acceptable excipients or carriers. In certain embodiments, the pharmaceutical compositions of the disclosure comprise a therapeutically effective amount of at least one compound of the disclosure and a pharmaceutically acceptable carrier. Pharmaceutically acceptable carriers, which are useful, include, but are not limited to, glycerol, water, saline, ethanol, recombinant human albumin (e.g., RECOMBUMIN®), solubilized gelatins (e.g., GELOFUSINE®), and other pharmaceutically acceptable salt solutions such as phosphates and salts of organic acids. Examples of these and other pharmaceutically acceptable carriers are described in Remington's Pharmaceutical Sciences (1991, Mack Publication Co., New Jersey).

The carrier may be a solvent or dispersion medium containing, for example, water, ethanol, polyol (for example, glycerol, propylene glycol, and liquid polyethylene glycol, and the like), recombinant human albumin, solubilized gelatins, suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In many cases, isotonic agents, for example, sugars, sodium chloride, or polyalcohols such as mannitol and sorbitol, are included in the composition. Prolonged absorption of the injectable compositions may be brought about by including in the composition an agent that delays absorption, for example, aluminum monostearate or gelatin.

Formulations may be employed in admixtures with conventional excipients, i.e., pharmaceutically acceptable organic or inorganic carrier substances suitable for oral, parenteral, nasal, inhalational, intravenous, subcutaneous, transdermal enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or fragrance-conferring substances and the like. They may also be combined where desired with other active agents, e.g., other analgesic, anxiolytics or hypnotic agents. As used herein, “additional ingredients” include, but are not limited to, one or more ingredients that may be used as a pharmaceutical carrier.

The composition of the disclosure may comprise a preservative from about 0.005% to 2.0% by total weight of the composition. The preservative is used to prevent spoilage in the case of exposure to contaminants in the environment. Examples of preservatives useful in accordance with the disclosure include but are not limited to those selected from the group consisting of benzyl alcohol, sorbic acid, parabens, imidurea and combinations thereof. One such preservative is a combination of about 0.5% to 2.0% benzyl alcohol and 0.05% to 0.5% sorbic acid.

The composition may include an antioxidant and a chelating agent which inhibit the degradation of the compound. Antioxidants for some compounds are BHT, BHA, alpha-tocopherol and ascorbic acid in the exemplary range of about 0.01% to 0.3%, or BHT in the range of 0.03% to 0.1% by weight by total weight of the composition. The chelating agent may be present in an amount of from 0.01% to 0.5% by weight by total weight of the composition. Exemplary chelating agents include edetate salts (e.g. disodium edetate) and citric acid in the weight range of about 0.01% to 0.20%, or in the range of 0.02% to 0.10% by weight by total weight of the composition. The chelating agent is useful for chelating metal ions in the composition that may be detrimental to the shelf life of the formulation. While BHT and disodium edetate are exemplary antioxidant and chelating agent, respectively, for some compounds, other suitable and equivalent antioxidants and chelating agents may be substituted therefore as would be known to those skilled in the art.

Liquid suspensions may be prepared using conventional methods to achieve suspension of the active ingredient in an aqueous or oily vehicle. Aqueous vehicles include, for example, water, and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin. Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent. Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, hydroxypropylmethyl cellulose. Known dispersing or wetting agents include, but are not limited to, naturally-occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g., polyoxyethylene stearate, heptadecaethyleneoxycetanol, polyoxyethylene sorbitol monooleate, and polyoxyethylene sorbitan monooleate, respectively). Known emulsifying agents include, but are not limited to, lecithin, acacia, and ionic or non-ionic surfactants. Known preservatives include, but are not limited to, methyl, ethyl, or n-propyl para-hydroxybenzoates, ascorbic acid, and sorbic acid. Known sweetening agents include, for example, glycerol, propylene glycol, sorbitol, sucrose, and saccharin.

Liquid solutions of the active ingredient in aqueous or oily solvents may be prepared in substantially the same manner as liquid suspensions, the primary difference being that the active ingredient is dissolved, rather than suspended in the solvent. As used herein, an “oily” liquid is one which comprises a carbon-containing liquid molecule and which exhibits a less polar character than water. Liquid solutions of the pharmaceutical composition of the disclosure may comprise each of the components described with regard to liquid suspensions, it being understood that suspending agents will not necessarily aid dissolution of the active ingredient in the solvent. Aqueous solvents include, for example, water, and isotonic saline. Oily solvents include, for example, almond oil, oily esters, ethyl alcohol, vegetable oils such as arachis, olive, sesame, or coconut oil, fractionated vegetable oils, and mineral oils such as liquid paraffin.

Powdered and granular formulations of a pharmaceutical preparation of the disclosure may be prepared using known methods. Such formulations may be administered directly to a subject, used, for example, to form tablets, to fill capsules, or to prepare an aqueous or oily suspension or solution by addition of an aqueous or oily vehicle thereto. Each of these formulations may further comprise one or more of dispersing or wetting agent, a suspending agent, ionic and non-ionic surfactants, and a preservative. Additional excipients, such as fillers and sweetening, flavoring, or coloring agents, may also be included in these formulations.

A pharmaceutical composition of the disclosure may also be prepared, packaged, or sold in the form of oil-in-water emulsion or a water-in-oil emulsion. The oily phase may be a vegetable oil such as olive or arachis oil, a mineral oil such as liquid paraffin, or a combination of these. Such compositions may further comprise one or more emulsifying agents such as naturally occurring gums such as gum acacia or gum tragacanth, naturally-occurring phosphatides such as soybean or lecithin phosphatide, esters or partial esters derived from combinations of fatty acids and hexitol anhydrides such as sorbitan monooleate, and condensation products of such partial esters with ethylene oxide such as polyoxyethylene sorbitan monooleate. These emulsions may also contain additional ingredients including, for example, sweetening or flavoring agents.

Methods for impregnating or coating a material with a chemical composition are known in the art, and include, but are not limited to methods of depositing or binding a chemical composition onto a surface, methods of incorporating a chemical composition into the structure of a material during the synthesis of the material (i.e., such as with a physiologically degradable material), and methods of absorbing an aqueous or oily solution or suspension into an absorbent material, with or without subsequent drying. Methods for mixing components include physical milling, the use of pellets in solid and suspension formulations and mixing in a transdermal patch, as known to those skilled in the art.

### Administration/Dosing

The regimen of administration may affect what constitutes an effective amount. The therapeutic formulations may be administered to the patient either prior to or after the onset of a disease or disorder. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions of the present disclosure to a patient, such as a mammal, such as a human, may be carried out using known procedures, at dosages and for periods of time effective to treat a disease or disorder contemplated herein. An effective amount of the therapeutic compound necessary to achieve a therapeutic effect may vary according to factors such as the activity of the particular compound employed; the time of administration; the rate of excretion of the compound; the duration of the treatment; other drugs, compounds or materials used in combination with the compound; the state of the disease or disorder, age, sex, weight, condition, general health and prior medical history of the patient being treated, and like factors well-known in the medical arts. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic compound of the disclosure is from about 0.01 mg/kg to 100 mg/kg of body weight/per day. One of ordinary skill in the art would be able to study the relevant factors and make the determination regarding the effective amount of the therapeutic compound without undue experimentation.

The compound may be administered to an animal as frequently as several times daily, or it may be administered less frequently, such as once a day, once a week, once every two weeks, once a month, or even less frequently, such as once every several months or even once a year or less. It is understood that the amount of compound dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on. The frequency of the dose is readily apparent to the skilled artisan and depends upon a number of factors, such as, but not limited to, type and severity of the disease being treated, and type and age of the animal.

Actual dosage levels of the active ingredients in the pharmaceutical compositions of this disclosure may be varied so as to obtain an amount of the active ingredient that is effective to achieve the desired therapeutic response for a particular patient, composition, and mode of administration, without being toxic to the patient.

A medical doctor, e.g., physician or veterinarian, having ordinary skill in the art may readily determine and prescribe the effective amount of the pharmaceutical composition required. For example, the physician or veterinarian could start doses of the compounds of the disclosure employed in the pharmaceutical composition at levels lower than that required in order to achieve the desired therapeutic effect and gradually increase the dosage until the desired effect is achieved.

In particular embodiments, it is especially advantageous to formulate the compound in dosage unit form for ease of administration and uniformity of dosage. Dosage unit form as used herein refers to physically discrete units suited as unitary dosages for the patients to be treated; each unit containing a predetermined quantity of therapeutic compound calculated to produce the desired therapeutic effect in association with the required pharmaceutical vehicle. The dosage unit forms of the disclosure are dictated by and directly dependent on (a) the unique characteristics of the therapeutic compound and the particular therapeutic effect to be achieved, and (b) the limitations inherent in the art of compounding/formulating such a therapeutic compound for the treatment of a disease or disorder in a patient.

In certain embodiments, the compositions of the disclosure are administered to the patient in dosages that range from one to five times per day or more. In other embodiments, the compositions of the disclosure are administered to the patient in range of dosages that include, but are not limited to, once every day, every two days, every three days to once a week, and once every two weeks. It will be readily apparent to one skilled in the art that the frequency of administration of the various combination compositions of the disclosure will vary from subject to subject depending on many factors including, but not limited to, age, disease or disorder to be treated, gender, overall health, and other factors. Thus, the disclosure should not be construed to be limited to any particular dosage regime and the precise dosage and composition to be administered to any patient will be determined by the attending physician taking all other factors about the patient into account.

Compounds of the disclosure for administration may be in the range of from about 1 μg to about 7,500 mg, about 20 μg to about 7,000 mg, about 40 μg to about 6,500 mg, about 80 μg to about 6,000 mg, about 100 μg to about 5,500 mg, about 200 μg to about 5,000 mg, about 400 μg to about 4,000 mg, about 800 μg to about 3,000 mg, about 1 mg to about 2,500 mg, about 2 mg to about 2,000 mg, about 5 mg to about 1,000 mg, about 10 mg to about 750 mg, about 20 mg to about 600 mg, about 30 mg to about 500 mg, about 40 mg to about 400 mg, about 50 mg to about 300 mg, about 60 mg to about 250 mg, about 70 mg to about 200 mg, about 80 mg to about 150 mg, and any and all whole or partial increments there-in-between.

In some embodiments, the dose of a compound of the disclosure is from about 0.5 μg and about 5,000 mg. In some embodiments, a dose of a compound of the disclosure used in compositions described herein is less than about 5,000 mg, or less than about 4,000 mg, or less than about 3,000 mg, or less than about 2,000 mg, or less than about 1,000 mg, or less than about 800 mg, or less than about 600 mg, or less than about 500 mg, or less than about 200 mg, or less than about 50 mg. Similarly, in some embodiments, a dose of a second compound as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

In certain embodiments, the present disclosure is directed to a packaged pharmaceutical composition comprising a container holding a therapeutically effective amount of a compound of the disclosure, alone or in combination with a second pharmaceutical agent; and instructions for using the compound to treat, prevent, or reduce one or more symptoms of a disease or disorder in a patient.

The term “container” includes any receptacle for holding the pharmaceutical composition or for managing stability or water uptake. For example, in certain embodiments, the container is the packaging that contains the pharmaceutical composition, such as liquid (solution and suspension), semisolid, lyophilized solid, solution and powder or lyophilized formulation present in dual chambers. In other embodiments, the container is not the packaging that contains the pharmaceutical composition, i.e., the container is a receptacle, such as a box or vial that contains the packaged pharmaceutical composition or unpackaged pharmaceutical composition and the instructions for use of the pharmaceutical composition. Moreover, packaging techniques are well known in the art. It should be understood that the instructions for use of the pharmaceutical composition may be contained on the packaging containing the pharmaceutical composition, and as such the instructions form an increased functional relationship to the packaged product. However, it should be understood that the instructions may contain information pertaining to the compound's ability to perform its intended function, e.g., treating, preventing, or reducing a disease or disorder in a patient.

### Administration

Routes of administration of any of the compositions of the disclosure include inhalational, oral, nasal, rectal, parenteral, sublingual, transdermal, transmucosal (e.g., sublingual, lingual, (trans)buccal, (trans)urethral, vaginal (e.g., trans- and perivaginally), (intra)nasal, and (trans)rectal), intravesical, intrapulmonary, intraduodenal, intragastrical, intrathecal, epidural, intrapleural, intraperitoneal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, emulsions, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present disclosure are not limited to the particular formulations and compositions that are described herein.

### Oral Administration

For oral application, particularly suitable are tablets, dragees, liquids, drops, capsules, caplets and gelcaps. Other formulations suitable for oral administration include, but are not limited to, a powdered or granular formulation, an aqueous or oily suspension, an aqueous or oily solution, a paste, a gel, toothpaste, a mouthwash, a coating, an oral rinse, or an emulsion. The compositions intended for oral use may be prepared according to any method known in the art and such compositions may contain one or more agents selected from the group consisting of inert, non-toxic, generally recognized as safe (GRAS) pharmaceutically excipients which are suitable for the manufacture of tablets. Such excipients include, for example an inert diluent such as lactose; granulating and disintegrating agents such as cornstarch; binding agents such as starch; and lubricating agents such as magnesium stearate.

Tablets may be non-coated or they may be coated using known methods to achieve delayed disintegration in the gastrointestinal tract of a subject, thereby providing sustained release and absorption of the active ingredient. By way of example, a material such as glyceryl monostearate or glyceryl distearate may be used to coat tablets. Further by way of example, tablets may be coated using methods described in U.S. Pat. Nos. 4,256,108; 4,160,452; and U.S. Pat. No. 4,265,874 to form osmotically controlled release tablets. Tablets may further comprise a sweetening agent, a flavoring agent, a coloring agent, a preservative, or some combination of these in order to provide for pharmaceutically elegant and palatable preparation. Hard capsules comprising the active ingredient may be made using a physiologically degradable composition, such as gelatin. The capsules comprise the active ingredient, and may further comprise additional ingredients including, for example, an inert solid diluent such as calcium carbonate, calcium phosphate, or kaolin.

Hard capsules comprising the active ingredient may be made using a physiologically degradable composition, such as gelatin. Such hard capsules comprise the active ingredient, and may further comprise additional ingredients including, for example, an inert solid diluent such as calcium carbonate, calcium phosphate, or kaolin.

Soft gelatin capsules comprising the active ingredient may be made using a physiologically degradable composition, such as gelatin from animal-derived collagen or from a hypromellose, a modified form of cellulose, and manufactured using optional mixtures of gelatin, water and plasticizers such as sorbitol or glycerol. Such soft capsules comprise the active ingredient, which may be mixed with water or an oil medium such as peanut oil, liquid paraffin, or olive oil.

For oral administration, the compounds of the disclosure may be in the form of tablets or capsules prepared by conventional means with pharmaceutically acceptable excipients such as binding agents; fillers; lubricants; disintegrates; or wetting agents. If desired, the tablets may be coated using suitable methods and coating materials such as OPADRY® film coating systems available from Colorcon, West Point, Pa. (e.g., OPADRY® OY Type, OYC Type, Organic Enteric OY-P Type, Aqueous Enteric OY-A Type, OY-PM Type and OPADRY® White, 32K18400). It is understood that similar type of film coating or polymeric products from other companies may be used.

A tablet comprising the active ingredient may, for example, be made by compressing or molding the active ingredient, optionally with one or more additional ingredients. Compressed tablets may be prepared by compressing, in a suitable device, the active ingredient in a free-flowing form such as a powder or granular preparation, optionally mixed with one or more of a binder, a lubricant, an excipient, a surface active agent, and a dispersing agent. Molded tablets may be made by molding, in a suitable device, a mixture of the active ingredient, a pharmaceutically acceptable carrier, and at least sufficient liquid to moisten the mixture. Pharmaceutically acceptable excipients used in the manufacture of tablets include, but are not limited to, inert diluents, granulating and disintegrating agents, binding agents, and lubricating agents. Known dispersing agents include, but are not limited to, potato starch and sodium starch glycolate. Known surface-active agents include, but are not limited to, sodium lauryl sulphate. Known diluents include, but are not limited to, calcium carbonate, sodium carbonate, lactose, microcrystalline cellulose, calcium phosphate, calcium hydrogen phosphate, and sodium phosphate. Known granulating and disintegrating agents include, but are not limited to, corn starch and alginic acid. Known binding agents include, but are not limited to, gelatin, acacia, pre-gelatinized maize starch, polyvinylpyrrolidone, and hydroxypropyl methylcellulose. Known lubricating agents include, but are not limited to, magnesium stearate, stearic acid, silica, and talc.

Granulating techniques are well known in the pharmaceutical art for modifying starting powders or other particulate materials of an active ingredient. The powders are typically mixed with a binder material into larger permanent free-flowing agglomerates or granules referred to as a “granulation.” For example, solvent-using “wet” granulation processes are generally characterized in that the powders are combined with a binder material and moistened with water or an organic solvent under conditions resulting in the formation of a wet granulated mass from which the solvent must then be evaporated.

Melt granulation generally consists in the use of materials that are solid or semi-solid at room temperature (i.e., having a relatively low softening or melting point range) to promote granulation of powdered or other materials, essentially in the absence of added water or other liquid solvents. The low melting solids, when heated to a temperature in the melting point range, liquefy to act as a binder or granulating medium. The liquefied solid spreads itself over the surface of powdered materials with which it is contacted, and on cooling, forms a solid granulated mass in which the initial materials are bound together. The resulting melt granulation may then be provided to a tablet press or be encapsulated for preparing the oral dosage form. Melt granulation improves the dissolution rate and bioavailability of an active (i.e., drug) by forming a solid dispersion or solid solution.

U.S. Pat. No. 5,169,645 discloses directly compressible wax-containing granules having improved flow properties. The granules are obtained when waxes are admixed in the melt with certain flow improving additives, followed by cooling and granulation of the admixture. In certain embodiments, only the wax itself melts in the melt combination of the wax(es) and additives(s), and in other cases both the wax(es) and the additives(s) will melt.

The present disclosure also includes a multi-layer tablet comprising a layer providing for the delayed release of one or more compounds useful within the methods of the disclosure, and a further layer providing for the immediate release of one or more compounds useful within the methods of the disclosure. Using a wax/pH-sensitive polymer mix, a gastric insoluble composition may be obtained in which the active ingredient is entrapped, ensuring its delayed release.

Liquid preparation for oral administration may be in the form of solutions, syrups or suspensions. The liquid preparations may be prepared by conventional means with pharmaceutically acceptable additives such as suspending agents (e.g., sorbitol syrup, methyl cellulose or hydrogenated edible fats); emulsifying agent (e.g., lecithin or acacia); non-aqueous vehicles (e.g., almond oil, oily esters or ethyl alcohol); and preservatives (e.g., methyl or propyl para-hydroxy benzoates or sorbic acid). Liquid formulations of a pharmaceutical composition of the disclosure which are suitable for oral administration may be prepared, packaged, and sold either in liquid form or in the form of a dry product intended for reconstitution with water or another suitable vehicle prior to use.

### Parenteral Administration

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intravenous, intraperitoneal, intramuscular, intrasternal injection, and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multidose containers containing a preservative. Injectable formulations may also be prepared, packaged, or sold in devices such as patient-controlled analgesia (PCA) devices. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e., powder or granular) form for reconstitution with a suitable vehicle (e.g., sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally acceptable diluent or solvent, such as water or 1,3-butanediol, for example. Other acceptable diluents and solvents include, but are not limited to, Ringer's solution, isotonic sodium chloride solution, and fixed oils such as synthetic mono- or di-glycerides. Other parentally-administrable formulations which are useful include those which comprise the active ingredient in microcrystalline form in a recombinant human albumin, a fluidized gelatin, in a liposomal preparation, or as a component of a biodegradable polymer system. Compositions for sustained release or implantation may comprise pharmaceutically acceptable polymeric or hydrophobic materials such as an emulsion, an ion exchange resin, a sparingly soluble polymer, or a sparingly soluble salt.

### Topical Administration

An obstacle for topical administration of pharmaceuticals is the stratum corneum layer of the epidermis. The stratum corneum is a highly resistant layer comprised of protein, cholesterol, sphingolipids, free fatty acids and various other lipids, and includes cornified and living cells. One of the factors that limit the penetration rate (flux) of a compound through the stratum corneum is the amount of the active substance that can be loaded or applied onto the skin surface. The greater the amount of active substance which is applied per unit of area of the skin, the greater the concentration gradient between the skin surface and the lower layers of the skin, and in turn the greater the diffusion force of the active substance through the skin. Therefore, a formulation containing a greater concentration of the active substance is more likely to result in penetration of the active substance through the skin, and more of it, and at a more consistent rate, than a formulation having a lesser concentration, all other things being equal.

Formulations suitable for topical administration include, but are not limited to, liquid or semi-liquid preparations such as liniments, lotions, oil-in-water or water-in-oil emulsions such as creams, ointments or pastes, and solutions or suspensions. Topically administrable formulations may, for example, comprise from about 1% to about 10% (w/w) active ingredient, although the concentration of the active ingredient may be as high as the solubility limit of the active ingredient in the solvent. Formulations for topical administration may further comprise one or more of the additional ingredients described herein.

Enhancers of permeation may be used. These materials increase the rate of penetration of drugs across the skin. Typical enhancers in the art include ethanol, glycerol monolaurate, PGML (polyethylene glycol monolaurate), dimethylsulfoxide, and the like. Other enhancers include oleic acid, oleyl alcohol, ethoxydiglycol, laurocapram, alkanecarboxylic acids, dimethylsulfoxide, polar lipids, or N-methyl-2-pyrrolidone.

One acceptable vehicle for topical delivery of some of the compositions of the disclosure may contain liposomes. The composition of the liposomes and their use are known in the art (i.e., U.S. Pat. No. 6,323,219).

In alternative embodiments, the topically active pharmaceutical composition may be optionally combined with other ingredients such as adjuvants, anti-oxidants, chelating agents, surfactants, foaming agents, wetting agents, emulsifying agents, viscosifiers, buffering agents, preservatives, and the like. In other embodiments, a permeation or penetration enhancer is included in the composition and is effective in improving the percutaneous penetration of the active ingredient into and through the stratum corneum with respect to a composition lacking the permeation enhancer. Various permeation enhancers, including oleic acid, oleyl alcohol, ethoxydiglycol, laurocapram, alkanecarboxylic acids, dimethylsulfoxide, polar lipids, or N-methyl-2-pyrrolidone, are known to those of skill in the art. In another aspect, the composition may further comprise a hydrotropic agent, which functions to increase disorder in the structure of the stratum corneum, and thus allows increased transport across the stratum corneum. Various hydrotropic agents such as isopropyl alcohol, propylene glycol, or sodium xylene sulfonate, are known to those of skill in the art.

The topically active pharmaceutical composition should be applied in an amount effective to affect desired changes. As used herein “amount effective” shall mean an amount sufficient to cover the region of skin surface where a change is desired. An active compound should be present in the amount of from about 0.0001% to about 15% by weight volume of the composition. For example, it should be present in an amount from about 0.0005% to about 5% of the composition; for example, it should be present in an amount of from about 0.001% to about 1% of the composition. Such compounds may be synthetically- or naturally derived.

### Buccal Administration

A pharmaceutical composition of the disclosure may be prepared, packaged, or sold in a formulation suitable for buccal administration. Such formulations may, for example, be in the form of tablets or lozenges made using conventional methods, and may contain, for example, 0.1 to 20% (w/w) of the active ingredient, the balance comprising an orally dissolvable or degradable composition and, optionally, one or more of the additional ingredients described herein. Alternately, formulations suitable for buccal administration may comprise a powder or an aerosolized or atomized solution or suspension comprising the active ingredient. Such powdered, aerosolized, or aerosolized formulations, when dispersed, may have an average particle or droplet size in the range from about 0.1 to about 200 micrometers, and may further comprise one or more of the additional ingredients described herein. The examples of formulations described herein are not exhaustive and it is understood that the disclosure includes additional modifications of these and other formulations not described herein, but which are known to those of skill in the art.

### Rectal Administration

A pharmaceutical composition of the disclosure may be prepared, packaged, or sold in a formulation suitable for rectal administration. Such a composition may be in the form of, for example, a suppository, a retention enema preparation, and a solution for rectal or colonic irrigation.

Suppository formulations may be made by combining the active ingredient with a non-irritating pharmaceutically acceptable excipient which is solid at ordinary room temperature (i.e., about 20° C.) and which is liquid at the rectal temperature of the subject (i.e., about 37° C. in a healthy human). Suitable pharmaceutically acceptable excipients include, but are not limited to, cocoa butter, polyethylene glycols, and various glycerides. Suppository formulations may further comprise various additional ingredients including, but not limited to, antioxidants, and preservatives.

Retention enema preparations or solutions for rectal or colonic irrigation may be made by combining the active ingredient with a pharmaceutically acceptable liquid carrier. As is well known in the art, enema preparations may be administered using, and may be packaged within, a delivery device adapted to the rectal anatomy of the subject. Enema preparations may further comprise various additional ingredients including, but not limited to, antioxidants, and preservatives.

### Additional Administration Forms

Additional dosage forms of this disclosure include dosage forms as described in U.S. Pat. Nos. 6,340,475, 6,488,962, 6,451,808, 5,972,389, 5,582,837, and 5,007,790. Additional dosage forms of this disclosure also include dosage forms as described in U.S. Patents Applications Nos. 20030147952, 20030104062, 20030104053, 20030044466, 20030039688, and 20020051820. Additional dosage forms of this disclosure also include dosage forms as described in PCT Applications Nos. WO 03/35041, WO 03/35040, WO 03/35029, WO 03/35177, WO 03/35039, WO 02/96404, WO 02/32416, WO 01/97783, WO 01/56544, WO 01/32217, WO 98/55107, WO 98/11879, WO 97/47285, WO 93/18755, and WO 90/11757.

### Controlled Release Formulations and Drug Delivery Systems:

In certain embodiments, the compositions and/or formulations of the present disclosure may be, but are not limited to, short-term, rapid-offset, as well as controlled, for example, sustained release, delayed release and pulsatile release formulations.

The term sustained release is used in its conventional sense to refer to a drug formulation that provides for gradual release of a drug over an extended period of time, and that may, although not necessarily, result in substantially constant blood levels of a drug over an extended time period. The period of time may be as long as a month or more and should be a release which is longer that the same amount of agent administered in bolus form.

For sustained release, the compounds may be formulated with a suitable polymer or hydrophobic material which provides sustained release properties to the compounds. As such, the compounds for use the method of the disclosure may be administered in the form of microparticles, for example, by injection or in the form of wafers or discs by implantation.

In certain embodiments of the disclosure, the compounds useful within the disclosure are administered to a subject, alone or in combination with another pharmaceutical agent, using a sustained release formulation.

The term delayed release is used herein in its conventional sense to refer to a drug formulation that provides for an initial release of the drug after some delay following drug administration and that may, although not necessarily, include a delay of from about 10 minutes up to about 12 hours.

The term pulsatile release is used herein in its conventional sense to refer to a drug formulation that provides release of the drug in such a way as to produce pulsed plasma profiles of the drug after drug administration.

The term immediate release is used in its conventional sense to refer to a drug formulation that provides for release of the drug immediately after drug administration.

As used herein, short-term refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes and any or all whole or partial increments thereof after drug administration after drug administration.

As used herein, rapid-offset refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes, and any and all whole or partial increments thereof after drug administration.

Those skilled in the art will recognize, or be able to ascertain using no more than routine experimentation, numerous equivalents to the specific procedures, embodiments, claims, and examples described herein. Such equivalents were considered to be within the scope of this disclosure and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application.

It is to be understood that, wherever values and ranges are provided herein, the description in range format is merely for convenience and brevity and should not be construed as an inflexible limitation on the scope of the disclosure. Accordingly, all values and ranges encompassed by these values and ranges are meant to be encompassed within the scope of the present disclosure. Moreover, all values that fall within these ranges, as well as the upper or lower limits of a range of values, are also contemplated by the present application. The description of a range should be considered to have specifically disclosed all the possible sub-ranges as well as individual numerical values within that range and, when appropriate, partial integers of the numerical values within ranges. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

The examples provided herein illustrate aspects of the present disclosure. However, they are in no way a limitation of the teachings or disclosure of the present disclosure as set forth herein. The examples herein are provided for the purpose of illustration only, and the disclosure is not limited to these examples, but rather encompasses all variations that are evident as a result of the teachings provided herein.

### EXAMPLES

The disclosure is now described with reference to the following Examples. These Examples are provided for the purpose of illustration only, and the disclosure is not limited to these Examples, but rather encompasses all variations that are evident as a result of the teachings provided herein.

### Step 1-Synthesis of 8,9-difluoropyrano[3,4-c]isochromene-1,6(2H,4H)-dione (B)

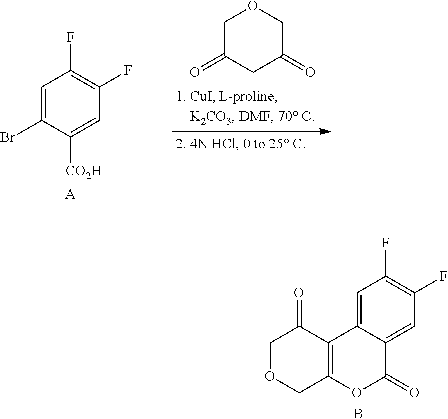

DMF (20.5 L) was added to a 50 L reactor and the contents were evacuated and backfilled with N₂ twice. Next, pyran-3,5-dione (1.28 kg, 11.22 mol, 1.2 eq.) was added, followed by 2-bromo-4,5-difluorobenzoic acid (A) (2.20 kg, 9.28 mol, 1.0 eq.), L-proline (214 g, 1.86 mol, 0.2 eq.), K₂CO₃ (2.59 kg, 18.6 mol, 2 eq.), and copper (I) iodide (177 g, 0.93 mol, 0.1 eq.). The mixture was again degassed and backfilled with N₂ two additional times. The reaction mixture was heated to 70° C. over the course of 1 h and was stirred at that temperature for no less than 10 h until completion of reaction was confirmed by LCMS. The reaction mixture was cooled to 5° C., after which it was diluted with water (20 L) over the course of 1.5 h, while maintaining the temperature below 12° C. The resulting black suspension was slowly acidified to pH 2 using 4 N HCl (7.5 L). Isopropyl acetate (4.5 L) was added, and the resulting mixture was warmed to 20° C. over the course of 1 h. The mixture was stirred at 20° C. as white solids precipitated for no less than 48 h, until completion of reaction as confirmed by LCMS (>90% of formation of B). The solids were filtered, and the wet cake was washed sequentially with water (2×3 L) and isopropyl acetate (2×3 L). After fully deliquoring, the wet cake was dried in vacuo at 40° C. for 16 h to provide compound (B) (1.659 kg, 71%) as an off-white solid. 1H NMR (400 MHZ, SO(CD3)₂) δ 8.65 (dd, J=7.8, 12.4 Hz, 1H), 8.21 (dd, J=8.0, 10.1 Hz, 1H), 4.80 (s, 2H), 4.33 (s, 2H); 13C NMR (100.6 MHz, SO(CD3)₂) δ: 192.4 (s), 168.4 (d, J=2.0 Hz), 157.0 (s), 154.2 (dd, J=13.2, 256.1 Hz), 149.1 (dd, J=14.0, 251.5 Hz), 130.9 (d, J=10.0 Hz), 118.3 (d, J=19.3 Hz), 117.6 (d, J=6.5 Hz), 113.6 (d, J=21.3 Hz), 107.3 (s), 71.4 (s), 64.2 (s); ¹⁹F NMR (376.5 MHz, SO(CD₃)₂) δ: −124.9 (ddd, J=8.5, 12.4, 22.0 Hz, 1F), −134.5 (dt, J=9.0, 23.3 Hz, 1F).

### Step 2-Synthesis of 8,9-difluoro-2H-pyrano[3,4-c]isoquinoline-1,6(4H,5H)-dione (C)

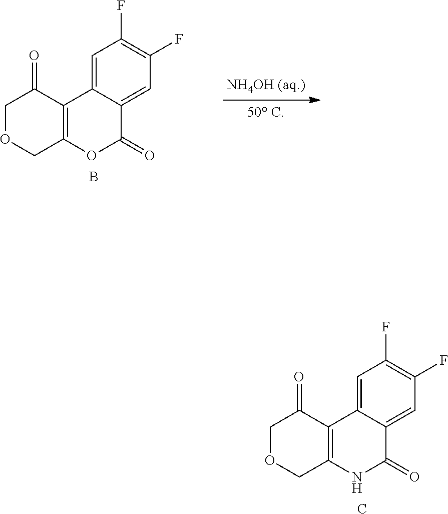

A 20 L reactor was charged with compound B (1.649 kg, 6.54 mole) and 28-30% NH₄OH (aq.) (6.27 L), and the resulting slurry was heated to 50° C. over the course of 45 min. The solid fully dissolved upon heating, and product began to precipitate after 10 min. After stirring for no less than 5 h at 50° C., completion of the reaction was confirmed by LCMS (disappearance of B and formation of C). The reaction mixture was cooled to 5° C. and stirred for 30 min. The cold slurry was filtered, and the wet cake was washed sequentially with water (3×1 L) and methanol (2×1 L). After fully deliquoring, the wet cake was dried in vacuo at 40° C. for 12 h to provide compound (C) (1.536 kg, 94%) as a white solid. 1H NMR (400 MHZ, SO(CD3)₂) δ: 12.35 (br s, 1H), 8.90 (dd, J=8.0, 13.2 Hz, 1H), 8.09 (dd, J=8.6, 10.8 Hz, 1H), 4.77 (s, 2H), 4.27 (s, 2H); 13C NMR (100.6 MHz, SO(CD3)₂) δ: 191.7 (s), 160.2 (s), 153.9 (s), 153.5 (dd, J=13.1, 251.5 Hz), 148.9 (dd, J=14.1, 249.5 Hz), 132.0 (d, J=9.1 Hz), 122.5 (s), 115.2 (d, J=17.1 Hz), 113.7 (d, J=20.1 Hz), 104.9 (s), 72.2 (s), 64.4 (s); ¹⁹F NMR (376.5 MHz, SO(CD3)₂) δ: −128.7 (ddd, J=8.5, 13.2, 22.4 Hz, 1F), −137.1 (ddd, J=8.0, 11.0, 23.6 Hz, 1F); MS (ESI−, m z): Calc'd for [C₁₂H₇F₂NO₃—H]⁻: 250.0; Found: 250.0.

### Step 3-Synthesis of (S)-8,9-difluoro-1-(((R)-1-phenylethyl)amino)-1,5-dihydro-2H-pyrano[3,4-c]isoquinolin-6(4H)-one (D)

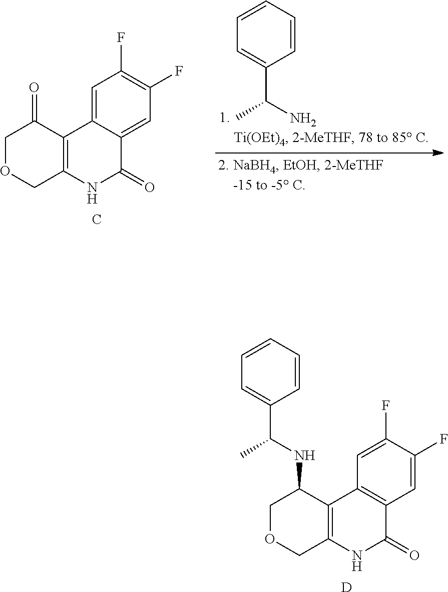

A 100 L reactor was charged with ketone C (670 g, 2.67 mol), (R)-1-phenylethan-1-amine (388 g, 3.20 mol, 1.2 eq.), and Ti(OEt)+(913 g, 4.00 mol, 1.5 eq.). Next, 2-MeTHF (6.7 L) was added, and the resulting suspension was heated to an internal temperature of 80° C. for no less than 21 h, until completion of the reaction, as confirmed by HPLC (formation of compound D after quenching an aliquot with NaBH₄ and EtOH). The reaction mixture was cooled to 50° C. and additional 2-MeTHF (13.4 L) was added. The resulting solution was cooled to −11° C., and EtOH (1.34 L) and NaBH+(151.4 g, 4.0 mol, 1.5 eq.) were alternately added in 4 portions over 1 h, while maintaining the reaction temperature below −8° C. The resulting mixture was stirred between −11° C. and −8° C. for no less than 5 h, until the reaction conversion was >99% by HPLC (formation of compound D).

The reaction mixture was quenched by addition into a pre-made solution of citric acid (3.86 kg) and NaOH (643 g) in water (20.1 L), while maintaining the temperature below 24° C. Additional 2-MeTHF (2.0 L) was used to quantitate the transfer. The resulting mixture was stirred at 24° C. for 12 h, during which period the mixture became two homogeneous layers. The organic layer was separated, and the aqueous layer was further extracted with 2-MeTHF (6.7 L). The combined organic layers were washed sequentially with water (6.7 L), 8% NaHCO₃ (aq.) (13.4 L), and water (10.0 L). The organic layer was diluted with 2-MeTHF (5.4 L) and was filtered through a filter paper.

The filtered solution was concentrated to a total volume of 5.4 L, during which some solids precipitated from the mixture. The mixture was diluted with 2-MeTHF (1.3 L) while rinsing the sides of the reaction vessel. The resulting slurry was stirred at 22° C. for 6.5 h, after which the mixture was heated to 40° C. over the course of 1.5 h. The mixture was agitated at 40° C. for 1.5 h, and was then cooled back to 22° C. over 1.5 h. The slurry was stirred at the same temperature for 3.5 h, after which the solid was filtered. The wet cake was washed with pre-cooled 2-MeTHF (0.67 L) at 0° C. and fully deliquored. The wet cake was dried in vacuo at 40° C. for 16 h to provide compound (D) (503.1 g, 53%) as a light tan powder. The yield of the isolation could be improved to >65% by using n-heptane as antisolvent during the crystallization. 1H NMR (400 MHz, SO(CD3)₂) δ: 11.31 (br s, 1H), 7.94 (dd, J=8.4, 11.0 Hz, 1H), 7.69 (dd, J=7.6, 12.4 Hz, 1H), 7.31-7.10 (m, 5H), 4.37 (d, J=16.1 Hz, 1H), 4.27 (d, J=16.1 Hz, 1H), 4.09 (q, J=6.5 Hz, 1H), 3.97 (d, J=11.8 Hz, 1H), 3.79 (s, 1H), 3.48 (dd, J=2.3, 11.7 Hz, 1H), 2.10 (br s, 1H), 1.34 (d, J=6.5 Hz, 3H); 13C NMR (100.6 MHz, SO(CD3)₂) δ: 159.8 (d, J=3.1 Hz), 152.5 (dd, J=14.1, 251 Hz), 147.9 (dd, J=14.2, 246 Hz), 146.9 (s), 136.7 (d, J=2.2 Hz), 135.6 (dd, J=2.4, 8.8 Hz), 128.1 (s), 126.6 (s), 126.4 (s), 122.4 (dd, J=1.7, 5.5 Hz), 114.6 (d, J=18.0 Hz), 111.2 (d, J=18.7 Hz), 107.3 (d, J=2.9 Hz), 67.2 (s), 63.4 (s), 54.8 (s), 47.2 (s), 23.7 (s); 19F NMR (376.5 MHz, SO(CD3)₂) δ: −131.6 (ddd, J=8.5, 12.4, 23.6 Hz, 1F), −139.9 (ddd, J=7.6, 11.0, 23.1 Hz, 1F); MS (ESI+, m/z): Calc'd for [C₂₀H₁₈F₂N₂O₂+H]⁺: 357.1; Found: 357.1.

### Step 3′-Synthesis of (S)-8,9-difluoro-1-(((R)-1-(4-methoxyphenyl)ethyl)amino)-1,5-dihydro-2H-pyrano[3,4-c]isoquinolin-6(4H)-one (D′)

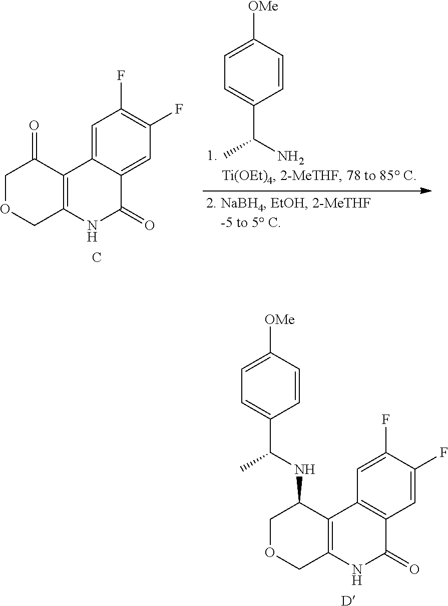

To a 50 L reactor was added ketone C (650 g, 2.59 mol), (R)-1-(4-methoxyphenyl)ethan-1-amine (470 g, 3.11 mol, 1.2 eq.), and Ti(OEt)+(890 g, 3.89 mol, 1.5 eq.). 2-MeTHF (6.5 L) was added and the resulting suspension was heated to a temperature of 80° C. for no less than 15 h until completion of reaction as confirmed by HPLC (formation of D′ after quenching an aliquot with NaBH₄ and EtOH). The reaction mixture was cooled to 50° C. and additional 2-MeTHF (13 L) was added. The suspension was heated to a temperature of 70° C. and the mixture became a clear solution.

The solution was cooled to 50° C. and ethanol (1.05 L, 834 g, 18.1 mol, 7 eq.) was added. The mixture was cooled to −2° C. and NaBH₄ (28.6 g, 778 mmol, 0.3 eq.) was added and the mixture was stirred for 30 min at a temperature of −2 to 0° C. Additional NaBH₄ (28.6 g, 778 mmol, 0.3 eq.) was added and the mixture was stirred for 30 min at the same temperature. A third portion of NaBH₄ (57.2 g, 1.55 mol, 0.6 eq.) was added and the mixture was stirred for 1.5 h at 0° C. Additional NaBH₄ (28.6 g, 778 mmol, 0.3 eq.) was added and the mixture was stirred for 30 min at 0° C. Ethanol (150 mL, 2.59 mol, 1 eq.) and NaBH₄ (28.6 g, 778 mmol, 0.3 eq., 1.8 eq. total) were added and the mixture was stirred at 0° C. for 30 min. The reaction mixture was stirred at 0° C. until completion of reaction as confirmed by HPLC (formation of D′).

The reaction mixture at 0° C. was quenched by controlled addition of an aqueous solution of sodium citrate (1.0 M citric acid/0.8 M NaOH buffer, 6.0 L), causing the temperature to increase to 24° C. The reaction mixture was stirred at room temperature for 12 h. The layers were separated, and the organic layer was washed sequentially with a 10% aqueous solution of NaCl (2×3 L), an 8% NaHCO₃ (aq.) (2.4 L), and water (3 L). Heptane (3 L) was added to the separated organic layer, causing an additional aqueous layer to separate. The organic layer was filtered through a sintered glass funnel and was concentrated by vacuum distillation to a volume of ˜6.5 L. The resulting red solution was heated to 35° C. and heptane (2.0 L) was added dropwise over 1.5 h. The resulting thin slurry was stirred at 35° C. for 2 h, after which it was cooled to 27° C. and stirred at that temperature for 30 min. The mixture was filtered, and the wet cake was washed with a 1:2 mixture of 2-MeTHF-heptane (2×650 mL). After fully deliquoring, the wet cake was dried in vacuo at 40° C. for 4 h to provide D′ (397 g, 40%) as an off-white solid. 1H NMR (400 MHZ, SO(CD3)₂) δ: 11.16 (br s, 1H), 7.97 (dd, J=8.5, 11.0 Hz, 1H), 7.70 (dd, J=7.6, 12.4 Hz, 1H), 7.20 (d, J=8.6 Hz, 2H), 6.77 (d, J=8.6 Hz, 2H), 4.37 (d, J=16.1 Hz, 1H), 4.28 (d, J=16.1 Hz, 1H), 4.05 (q, J=6.5 Hz, 1H), 3.97 (d, J=11.6 Hz, 1H), 3.78 (s, 1H), 3.68 (s, 3H), 3.48 (dd, J=2.4, 11.7 Hz, 1H), 1.98 (br s, 1H), 1.30 (d, J=6.4 Hz, 3H); 13C NMR (100.6 MHz, SO(CD3)₂) δ: 159.9 (d, J=3.0 Hz), 158.0 (s), 152.6 (dd, J=14.1, 251.0 Hz), 148.1 (dd, J=14.3, 246.9 Hz), 138.9 (s), 136.7 (d, J=2.2 Hz), 135.6 (d, J=8.8 Hz), 127.4 (s), 122.4 (d, J=5.4 Hz), 114.6 (d, J=18.0 Hz), 113.5 (s), 111.2 (d, J=18.7 Hz), 107.4 (d, J=2.8 Hz), 67.2 (s), 63.5 (s), 55.0 (d, J=1.6 Hz), 54.1 (s), 47.2 (s), 23.7 (s); 1° F. NMR (376.5 MHZ, SO(CD3)₂) δ: −131.6 (ddd, J=8.5, 12.4, 23.4 Hz, 1F), −139.9 (ddd, J=7.6, 11.1, 23.0 Hz, 1F); MS (ESI+, m/z): Calc'd for [C₂₁H₂₀F₂N₂O₃+H]⁺: 387.2; Found: 387.2.

### Step 4-Synthesis of (S)-8,9-difluoro-1-(methyl((R)-1-phenylethyl)amino)-1,5-dihydro-2H-pyrano[3,4-c]isoquinolin-6(4H)-one (E)

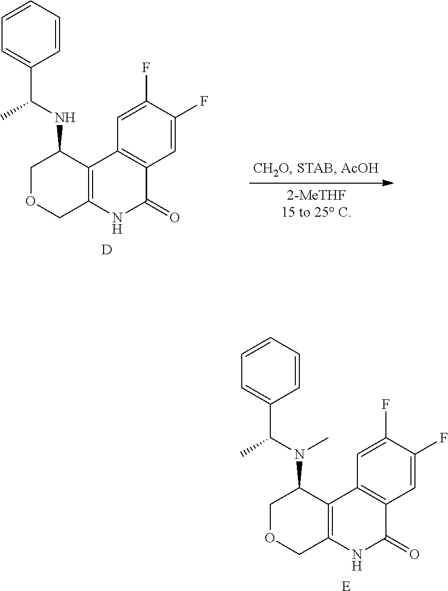

To a 20 L reactor was added amine D (480.0 g, 1.347 mol) and the solid was suspended in 2-MeTHF (9.6 L). Acetic acid (97.1 g, 1.62 mol, 1.2 eq.) and 37% formaldehyde (273.3 g, 101 g of formaldehyde, 3.37 mol, 2.5 eq.) were then added. The resulting suspension was cooled to 16° C. and NaBH(OAc)₃ (571 g, 2.69 mol, 2.0 eq.) was added in 4 portions over 25 min, maintaining the temperature below 23° C. The reaction mixture was stirred at 21° C. for NLT 3 h until completion of reaction as confirmed by HPLC (>99% conversion of D and formation of E). The mixture was quenched with 1 M NaOH (aq.) (4.8 L) over the course of 20 min and was then diluted with water (3.4 L). The resulting suspension was stirred at room temperature until the solids were completely dissolved. The organic layer was separated, and the aqueous layer was further extracted with 2-MeTHF (4.8 L). The combined organic layers were washed with water (4.8 L) and were dried over Na₂SO₄ (0.96 kg) for 1 h. The mixture was filtered, and the filtered solution was concentrated by vacuum distillation to a total volume of 7.2 L. Cyclopentyl methyl ether (CPME, 4.8 L) was added, and the mixture was concentrated to 7.2 L as solids precipitated. Additional CPME (4.8 L) was added and the mixture was concentrated to a total volume of 5.8 L. Additional CPME (1.4 L) was added and used to rinse the sides of the reaction vessel. The resulting slurry was stirred at 21° C. for 2 h, and the solids were filtered. The wet cake was washed sequentially with CPME (0.96 L) and n-heptane (2× 0.96 L), then fully deliquored. The wet cake was dried in vacuo at 55° C. to provide compound (E) (454.0 g, 449.5 g corrected for residual n-heptane and CPME, 90%) as an off-white solid. 1H NMR (400 MHZ, SO(CD3)₂) δ: 11.46 (s, 1H), 8.02 (dd, J=8.5, 10.8 Hz, 1H), 7.98 (dd, J=7.8, 12.7 Hz, 1H), 7.28-7.11 (m, 5H), 4.44 (d, J=16.1 Hz, 1H), 4.29 (d, J=16.1 Hz, 1H), 4.26 (d, J=12.8 Hz, 1H), 4.12 (s, 1H), 3.95 (q, J=6.7 Hz, 1H), 3.54 (dd, J=3.4, 12.4 Hz, 1H), 2.05 (s, 3H), 1.41 (d, J=6.7 Hz, 3H); 19F NMR (376.5 MHz, SO(CD3)₂) δ: −131.5 (ddd, J=8.6, 12.8, 22.2 Hz, 1F), −139.4 (ddd, J=7.9, 11.1, 23.1 Hz, 1F); MS (ESI+, m/z): Calc'd for [C₂₁H₂₀F₂N₂O₂+H]⁺: 371.2; Found: 371.2.

### Step 4′-Synthesis of (S)-8,9-difluoro-1-(((R)-1-(4-methoxyphenyl)ethyl)(methyl)amino)-1,5-dihydro-2H-pyrano[3,4-c]isoquinolin-6(4H)-one (E′)

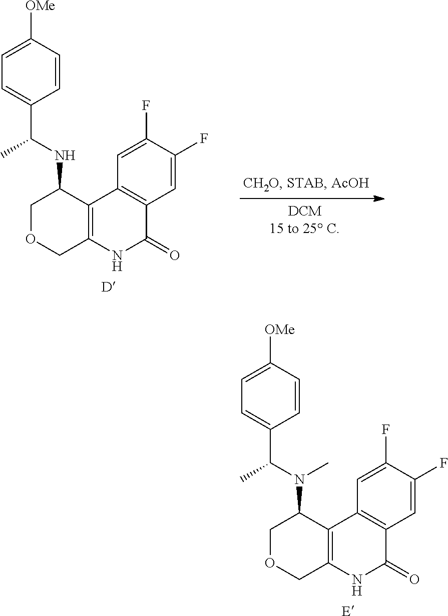

To a 20 L reactor was added D′ (803.5 g, 2.079 mol) and the solids were suspended in CH₂Cl₂ (8.8 L). The suspension was cooled to a temperature of 13° C. and acetic acid (143 mL, 150 g, 2.50 mol, 1.2 eq.) and 37% formaldehyde (422 g, 156 g of formaldehyde, 5.20 mol, 2.5 eq.) were added. NaBH(OAc)₃ (882 g, 4.19 mol, 2.0 eq.) was added in 6 portions over the course of 1 h, maintaining the temperature below 18° C. After the final addition, the solution was stirred at 12-14° C. for no less than 1 h until completion of reaction as confirmed by HPLC (disappearance of D′ and formation of E′). The reaction mixture was quenched portion-wise with 3 M NaOH (4 L). The resulting mixture was stirred for 20 min, and CH₂Cl₂ (4 L) was added, followed by water (1 L). The layers were separated, and the organic layer was washed with additional water (4 L). The organic layer was filtered through a pad of anhydrous Na₂SO₄, after which it was concentrated by vacuum distillation to a total volume of 5 L. Organic layer containing compound (E′) was taken as is to next step (Step 5′).

### Step 5-Synthesis of (S)-8,9-difluoro-1-(methylamino)-1,5-dihydro-2H-pyrano[3,4-c]isoquinolin-6(4H)-one trifluoroacetate salt (F)

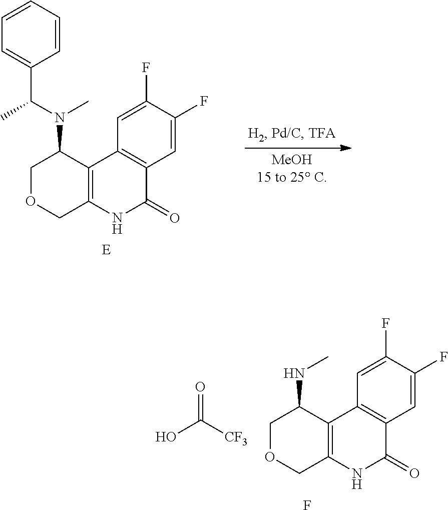

To a 20 L autoclave reactor was added compound E (420.0 g, 1.134 mol) and the solids were suspended in methanol (4.2 L). Trifluoroacetic acid (420 mL) was added and the mixture was stirred at room temperature until the solids fully dissolved. The mixture was degassed and purged with N₂ for 5 min, and 10% Pd/C (wet, 12.1 g as is, 1.21 g of Pd, 11.4 mmol, 0.01 eq.) was added. The suspension was degassed and purged with hydrogen gas for 5 min, and the mixture was backfilled with hydrogen gas (0.8 bar). This purge and backfill procedure was repeated two more times. The reaction mixture was stirred under a hydrogen atmosphere (0.8 bar) for no less than 3.5 h until completion of reaction as confirmed by HPLC (>99% conversion of E and formation of F). The mixture was purged with N₂ for 5 min, and the headspace of the reactor was backfilled with N₂. This purge and backfilling procedure was repeated three additional times. The reaction mixture was filtered through a CELITE® pad, and the solids were washed with methanol (2.1 L). The filtrate was concentrated by vacuum distillation to a total volume of 2.1 L, after which 2-MeTHF (8.4 L) was added. The mixture was concentrated to a total volume of 6.3 L and, as solids precipitated, additional 2-MeTHF (8.4 L) was added. The slurry was concentrated to a total volume of 5.5 L, and additional 2-MeTHF (0.8 L) was used to rinse the sides of the reaction vessel. The suspension was stirred at room temperature for 2 h, after which the solids were filtered, and the wet cake was washed with 2-MeTHF (2×0.65 L). After fully deliquoring, the wet cake was dried in vacuo at 50° C. to provide compound (F) (462.9 g, 411.5 g corrected for residual 2-MeTHF, 95%) as a white solid. 1H NMR (400 MHZ, SO(CD3)₂) δ: 11.82 (br s, 1H), 9.04 (br s, 2H), 8.08 (dd, J=8.4, 10.8 Hz, 1H), 8.02 (dd, J=7.3, 12.3 Hz, 1H), 4.64-4.51 (m, 3H), 4.46 (d, J=12.8 Hz, 1H), 3.89 (dd, J=1.8, 12.9 Hz, 1H), 2.66 (s, 3H); 13C NMR (100.6 MHz, SO(CD3)₂) δ: 159.9 (d, J=2.9 Hz), 158.3 (q, J=31.2 Hz), 153.0 (dd, J=13.9, 252.2 Hz), 148.5 (dd, J=14.4, 248.4 Hz), 140.0 (d, J=2.2 Hz), 133.9 (dd, J=2.4, 8.6 Hz), 122.2 (dd, J=2.2, 6.1 Hz), 117.2 (q, J=299.6 Hz), 115.2 (d, J=18.2 Hz), 111.6 (d, J=19.4 Hz), 100.9 (s), 63.9 (s), 62.5 (s), 48.6 (s), 30.5 (s); 1° F. NMR (376.5 MHZ, SO(CD3)₂) δ: −73.7 (s, 3F), −130.5 (ddd, J=8.4, 12.2, 22.9 Hz, 1F), −138.7 (ddd, J=7.3, 10.7, 22.9 Hz, 1F); MS (ESI+, m/z): Calc'd for [C₁₃H₁₂F₂N₂O₂+H]⁺: 267.1; Found: 267.1.

### Step 5′-Synthesis of (S)-8,9-difluoro-1-(methylamino)-1,5-dihydro-2H-pyrano[3,4-c]isoquinolin-6(4H)-one trifluoroacetate salt (F)

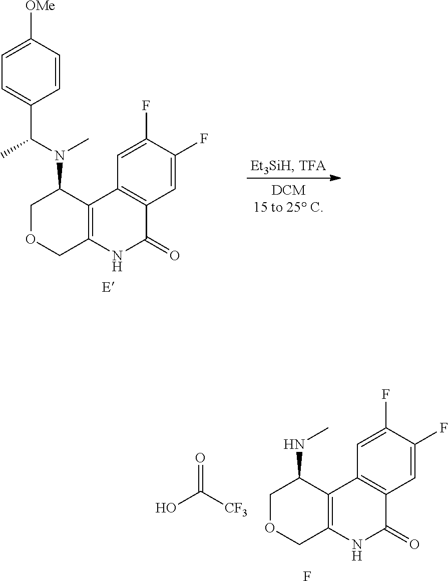

To a solution of E′ was added triethylsilane (484 g, 4.16 mol, 2 eq.), followed by trifluoroacetic acid (3.7 L). The resulting solution was stirred at room temperature for no less than 35 h until completion of reaction as confirmed by HPLC (disappearance of E′ and formation of F). The reaction mixture was distilled under reduced pressure to a total volume of 5 L. Methanol (1 L) was added and the mixture was further distilled to a volume of 5 L, as some solids began to precipitate. The thin slurry was diluted with a mixture of 2-MeTHF/methanol (4 L, 3:1 v v), causing the solid to dissolve. The solution was concentrated to a volume of 7 L, and additional 2-MeTHF/methanol (2 L, 3:1 v v) was added. The solution was concentrated to a volume of 8 L. 2-MeTHF (2 L) was added and the mixture was concentrated back to 8 L total volume as solids began to precipitate. This process was repeated two additional times. The resulting slurry was adjusted to a temperature of 27° C. and the solids were filtered. After deliquoring, the wet cake was washed with 2-MeTHF (1 L, then 2×0.5 L). After fully deliquoring, the wet cake was dried in vacuo at 40° C. for 24 h to provide compound (F) (673 g, 628 g corrected for residual 2-MeTHF, 79%) as a white solid. In certain embodiments, the yield is improved by performing additional solvent swaps to ensure complete removal of methanol before filtration. 1H NMR (400 MHZ, SO(CD3)₂) δ: 11.82 (br s, 1H), 9.04 (br s, 2H), 8.08 (dd, J=8.4, 10.8 Hz, 1H), 8.02 (dd, J=7.3, 12.3 Hz, 1H), 4.64-4.51 (m, 3H), 4.46 (d, J=12.8 Hz, 1H), 3.89 (dd, J=1.8, 12.9 Hz, 1H), 2.66 (s, 3H); 13C NMR (100.6 MHz, SO(CD3)₂) δ: 159.9 (d, J=2.9 Hz), 158.3 (q, J=31.2 Hz), 153.0 (dd, J=13.9, 252.2 Hz), 148.5 (dd, J=14.4, 248.4 Hz), 140.0 (d, J=2.2 Hz), 133.9 (dd, J=2.4, 8.6 Hz), 122.2 (dd, J=2.2, 6.1 Hz), 117.2 (q, J=299.6 Hz), 115.2 (d, J=18.2 Hz), 111.6 (d, J=19.4 Hz), 100.9 (s), 63.9 (s), 62.5 (s), 48.6 (s), 30.5 (s); 19F NMR (376.5 MHz, SO(CD3)₂) δ: −73.7 (s, 3F), −130.5 (ddd, J=8.4, 12.2, 22.9 Hz, 1F), −138.7 (ddd, J=7.3, 10.7, 22.9 Hz, 1F); MS (ESI+, m/z): Calc'd for [C₁₃H₁₂F₂N₂O₂+H]+: 267.1; Found: 267.1.

### Step 6-Synthesis of (S)—N-(8,9-difluoro-6-oxo-1,4,5,6-tetrahydro-2H-pyrano[3,4-c]isoquinolin-1-yl)-5,6-difluoro-N-methyl-1H-indole-2-carboxamide (X)

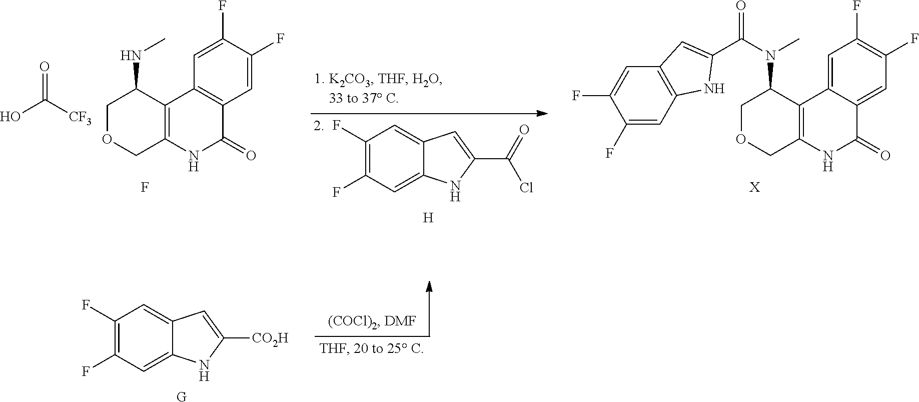

To a 10 L reactor were 5,6-difluoro-1H-indole-2-carboxylic acid (G) (159.7 g, 0.81 mol, 1.1 eq.) and DMF (5.4 g, 73.9 mmol, 0.1 eq.), and the contents were dissolved in THF (3.36 L). The resulting solution was cooled to 3° C. and oxalyl chloride (102.8 g, 0.81 mol, 1.1 eq.) was added over the course of 12 min. The solution was stirred at 3° C. for 1 h, after which the temperature was adjusted to 22° C. and the mixture was stirred at that temperature for 30 min. Additional oxalyl chloride (4.7 g, 37 mmol, 0.05 eq.) was added and the solution was stirred at room temperature for a further 30 min until reaction completion as confirmed by HPLC (disappearance of 5,6-difluoro-1H-indole-2-carboxylic acid and formation of the corresponding methyl ester after quenching with 5% sodium methoxide in methanol).

To a separate 30 L reactor was added compound F (280.0 g, 0.736 mol) and the solids were dissolved in a mixture of water (1.4 L) and THF (7.0 L). A solution of K₂CO₃ (305.3 g, 2.21 mol, 3 eq.) in water (0.56 L) was added and the mixture was heated to 32° C. The acid chloride (H) solution prepared as described herein was added to this mixture over 7 min, and THF (0.84 L) was used to quantitate the transfer. The reaction mixture was stirred at 35° C. for no less than 45 min, after which HPLC indicated >99% conversion (conversion of F and formation of X). The mixture was diluted with water (0.84 L) and ethyl acetate (5.6 L) and the contents were agitated at 35° C. for 20 min. The layers were separated, and the organic layer was washed with water (2.8 L), maintaining an internal temperature of 34° C. for 20 min. The layers were separated, and the organic layer was filtered through a CELITE® pad. The solids were washed with THF (1.4 L), and the resulting filtrate was cooled to 12° C. A small amount of solid precipitated from the solution, and the mixture was filtered through a filter paper. The solution was concentrated by vacuum distillation to a total volume of 8.4 L, after which ethanol (8.4 L) was added. The solution was concentrated to a total volume of 8.4 L, and additional ethanol (5.6 L) was added. The mixture was concentrated to a volume of 4.2 L as solids precipitated. Ethanol (5.6 L) was added, and the slurry was concentrated to a volume of 3.9 L. Additional ethanol (0.3 L) was used to rinse the sides of the reaction vessel. Water (0.42 L) was added, and the temperature of the slurry was adjusted to 38° C. The suspension was agitated at the same temperature for 1 h, after which it was cooled to 22° C. and stirred at that temperature for 2 h. The mixture was filtered, and the wet cake was washed with ethanol (0.76 L). After fully deliquoring, the wet cake was dried in vacuo at 60° C. to provide compound (X) (316.0 g, 310.9 g corrected for residual ethanol and water, 95%) as a white solid. 1H NMR (400 MHZ, SO(CD3)₂) δ: 11.92 (br s, 1H), 11.74 (br s, 1H), 8.12 (dd, J=8.4, 10.8 Hz, 1H), 7.61 (dd, J=8.0, 11.1 Hz, 1H), 7.43 (dd, J=7.5, 12.0 Hz, 1H), 7.38 (dd, J=7.2, 11.1 Hz, 1H), 6.95 (s, 1H), 5.73 (app s, 1H), 4.62 (d, J=16.2 Hz, 1H), 4.46 (d, J=15.7 Hz, 1H), 4.15 (d, J=12.2 Hz, 1H), 4.02 (dd, J=3.4, 12.3 Hz, 1H), 3.13 (s, 3H); 13C NMR (100.6 MHz, SO(CD3)₂) δ: 162.3 (s), 160.0 (d, J=2.9 Hz), 153.0 (dd, J=13.9, 252 Hz), 148.4 (dd, J=14.2, 248 Hz), 148.2 (dd, J=16.3, 241 Hz), 145.7 (dd, J=15.2, 236 Hz), 139.8 (s), 134.7 (d, J=8.3 Hz), 131.6 (d, J=3.6 Hz), 131.3 (d, J=11.3 Hz), 123.0 (d, J=5.9 Hz), 122.3 (d, J=8.6 Hz), 115.4 (d, J=18.3 Hz), 109.9 (d, J=19.0 Hz), 108.0 (s), 102.2 (s), 99.7 (d, J=21.4 Hz), 70.6 (s), 63.5 (s), 45.6 (s), 33.6 (s); 19F NMR (376.5 MHz, SO(CD3)₂) δ: −129.7 (dt, J=10.1, 21.4 Hz), −138.8 (dt, J=9.1, 20.8 Hz), —141.3 (ddd, J=8.1, 11.1, 21.9 Hz), −147.1 (ddd, J=7.0, 10.9, 21.6 Hz); MS (ESI+, m/z): Calc'd for [C₂₂H₁₅F₄N₃O₃+H]+: 446.1; Found: 446.1.

### ENUMERATED EMBODIMENTS

The following exemplary embodiments are provided, the numbering of which is not to be construed as designating levels of importance:

Embodiment 1 provides a method of preparing (S)—N-(8,9-difluoro-6-oxo-1,4,5,6-tetrahydro-2H-pyrano[3,4-c]isoquinolin-1-yl)-5,6-difluoro-N-methyl-1H-indole-2-carboxamide (X), or a salt or solvate thereof:

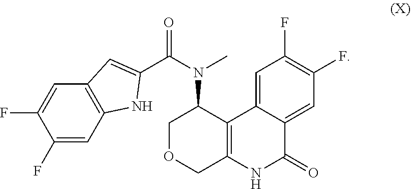 [SMILES: CN(C(=O)c1cc2cc(F)c(F)cc2[nH]1)[C@@H]1COCc2[nH]c(=O)c3cc(F)c(F)cc3c21]

the method comprising reacting (S)-8,9-difluoro-1-(methylamino)-1,5-dihydro-2H-pyrano[3,4-c]isoquinolin-6(4H)-one (F):

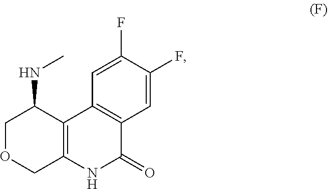 [SMILES: CN[C@@H]1COCc2[nH]c(=O)c3cc(F)c(F)cc3c21]

and 5,6-difluoro-1H-indole-2-carbonyl chloride (H):

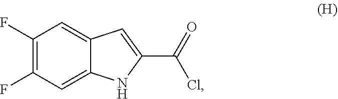 [SMILES: O=C(Cl)c1cc2cc(F)c(F)cc2[nH]1]

so as to generate a first reaction system comprising (X).

Embodiment 2 provides the method of Embodiment 1, wherein the reaction of (F) and (H) is performed in the presence of a base.

Embodiment 3 provides the method of Embodiment 2, wherein the base comprises K₂CO₃.

Embodiment 4 provides the method of any one of Embodiments 1-3, wherein (F) and (H) are reacted in a solvent comprising at least one of THE and H₂O.

Embodiment 5 provides the method of any one of Embodiments 1-4, wherein purification of (X) comprises adding at least one organic solvent to the first reaction system to generate a biphasic system.

Embodiment 6 provides the method of Embodiment 5, wherein the organic solvent comprises ethyl acetate.

Embodiment 7 provides the method of any one of Embodiments 5-6, wherein the purification of (X) further comprises separating the biphasic system to provide an aqueous phase and an organic phase.

Embodiment 8 provides the method of Embodiment 7, wherein the purification of (X) further comprises exchanging at least a fraction of the organic phase with ethanol to generate a first solution.

Embodiment 9 provides the method of Embodiment 8, wherein the purification of (X) further comprises adding water to the first solution to provide a second solution.

Embodiment 10 provides the method of Embodiment 9, wherein the purification of (X) further comprises heating and cooling the second solution to yield solid (X).

Embodiment 11 provides the method of any one of Embodiments 1-10, wherein (H) is prepared by reacting 5,6-difluoro-1H-indole-2-carboxylic acid (G) and a chlorinating agent.

Embodiment 12 provides the method of Embodiment 11, wherein the chlorinating agent is selected from the group consisting of (COCl)₂ and SOCl₂.

Embodiment 13 provides the method of any one of Embodiments 1-10, wherein (F) is prepared by reacting (S)-8,9-difluoro-1-(methyl((R)-1-phenylethyl)amino)-1,5-dihydro-2H-pyrano[3,4-c]isoquinolin-6(4H)-one (E) with hydrogen gas (H₂) in the presence of a hydrogenation catalyst and an acid:

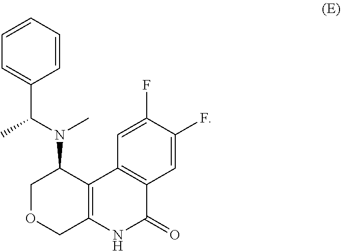 [SMILES: C[C@H](c1ccccc1)N(C)[C@@H]1COCc2[nH]c(=O)c3cc(F)c(F)cc3c21]

Embodiment 14 provides the method of Embodiment 13, wherein the hydrogenation catalyst comprises palladium on carbon (Pd/C).

Embodiment 15 provides the method of any one of Embodiments 13-14, wherein the acid comprises trifluoroacetic acid (TFA).

Embodiment 16 provides the method of any one of Embodiments 13-15, wherein (E) and hydrogen gas are reacted in a solvent comprising methanol.

Embodiment 17 provides the method of any one of Embodiments 13-16, wherein purification of (F) comprises exchanging at least a fraction of the methanol with 2-methyltetrahydrofuran, thereby generating a first solution.

Embodiment 18 provides the method of Embodiment 17, wherein at least partial concentration of the first solution yields solid (F).

Embodiment 19 provides the method of any one of Embodiments 13-18, wherein (E) is prepared by reductive alkylation of (S)-8,9-difluoro-1-(((R)-1-phenylethyl)amino)-1,5-dihydro-2H-pyrano[3,4-c]isoquinolin-6(4H)-one (D) with formaldehyde:

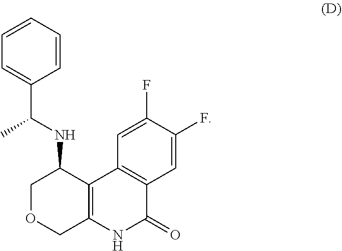 [SMILES: C[C@@H](N[C@@H]1COCc2[nH]c(=O)c3cc(F)c(F)cc3c21)c1ccccc1]

Embodiment 20 provides the method of Embodiment 19, wherein the reductive alkylation of the (D) is performed in a solvent comprising 2-methyltetrahydrofuran.

Embodiment 21 provides the method of any one of Embodiments 19-20, wherein (D) is prepared by reacting (R)-1-phenylethan-1-amine:

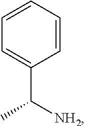

and 8,9-difluoro-2H-pyrano[3,4-c]isoquinoline-1,6(4H,5H)-dione (C):

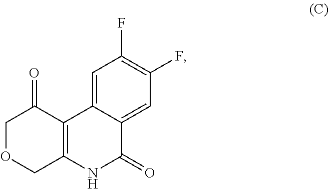 [SMILES: O=C1COCc2[nH]c(=O)c3cc(F)c(F)cc3c21]

so as to form an imine intermediate, and treating the imine intermediate with a reducing agent to form (D).

Embodiment 22 provides the method of Embodiment 21, wherein (C) and (R)-1-phenylethan-1-amine are reacted in the presence of a titanium (IV) alkoxide to form the imine intermediate.

Embodiment 23 provides the method of Embodiment 22, wherein the titanium (IV) alkoxide comprises Ti(OEt)₄.

Embodiment 24 provides the method of any one of Embodiments 21-23, wherein the reducing agent comprises a borohydride.

Embodiment 25 provides the method of any one of Embodiments 1-10, wherein (F) is prepared by reacting (S)-8,9-difluoro-1-(((R)-1-(4-methoxyphenyl)ethyl)(methyl)amino)-1,5-dihydro-2H-pyrano[3,4-c]isoquinolin-6(4H)-one (E′) with an acid:

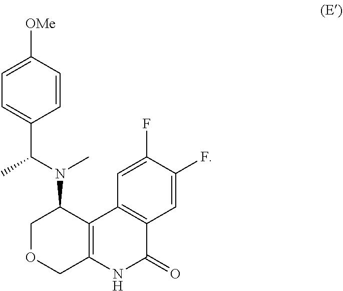 [SMILES: COc1ccc([C@@H](C)N(C)[C@@H]2COCc3[nH]c(=O)c4cc(F)c(F)cc4c32)cc1]

Embodiment 26 provides the method of Embodiment 25, wherein the acid comprises trifluoroacetic acid (TFA).

Embodiment 27 provides the method of any one of Embodiments 25-26, wherein (E′) and the acid are reacted in the presence of a trialkylsilane.

Embodiment 28 provides the method of any one of Embodiments 25-27, wherein (E′) is prepared by reductive alkylation of (S)-8,9-difluoro-1-(((R)-1-(4-methoxyphenyl)ethyl)amino)-1,5-dihydro-2H-pyrano[3,4-c]isoquinolin-6(4H)-one (D′) with formaldehyde:

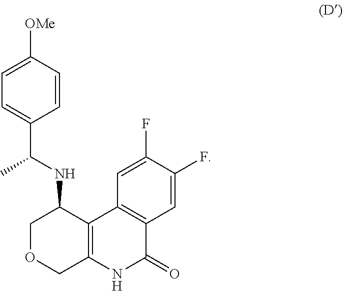 [SMILES: COc1ccc([C@@H](C)N[C@@H]2COCc3[nH]c(=O)c4cc(F)c(F)cc4c32)cc1]

Embodiment 29 provides the method of Embodiment 28, wherein (D′) is prepared by reacting (R)-1-(4-methoxyphenyl)ethan-1-amine:

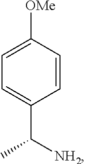

and 8,9-difluoro-2H-pyrano[3,4-c]isoquinoline-1,6(4H,5H)-dione (C):

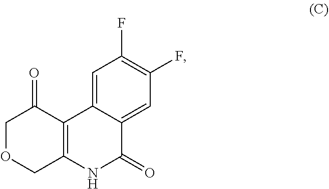 [SMILES: O=C1COCc2[nH]c(=O)c3cc(F)c(F)cc3c21]

so as to form an imine intermediate, and treating the imine intermediate with a reducing agent to form (D′).

Embodiment 30 provides the method of Embodiment 29, wherein (C) and the (R)-1-(4-methoxyphenyl)ethan-1-amine are reacted in the presence of a titanium (IV) alkoxide to form the imine.

Embodiment 31 provides the method of any one of Embodiments 29-30, wherein the reducing agent comprises a borohydride.

Embodiment 32 provides the method of any one of Embodiments 21-24 and 29-31, wherein the (C) is prepared by reacting 8,9-difluoropyrano[3,4-c]isochromene-1,6(2H,4H)-dione (B):

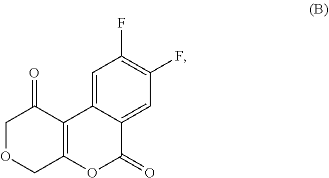 [SMILES: O=C1COCc2oc(=O)c3cc(F)c(F)cc3c21]

with an ammonium salt.

Embodiment 33 provides the method of Embodiment 32, wherein the ammonium salt comprises ammonium hydroxide.

Embodiment 34 provides the method of Embodiment 33, wherein the ammonium hydroxide comprises an aqueous solution.

Embodiment 35 provides the method of any one of Embodiments 32-34, wherein the reaction of (B) and the ammonium salt occurs at a temperature of about 50° C.

Embodiment 36 provides the method of any one of Embodiments 32-35, wherein (B) is prepared by reacting 2-bromo-4,5-difluorobenzoic acid (A):

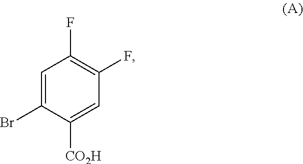 [SMILES: O=C(O)c1cc(F)c(F)cc1Br]

with 3,5-pyrandione so as to form a keto-acid intermediate, and treating the keto-acid intermediate with an acid to form (B).

Embodiment 37 provides the method of Embodiment 36, wherein (A) and 3,5-pyrandione are reacted in the presence of a Lewis acid.

Embodiment 38 provides the method of Embodiment 37, wherein the Lewis acid comprises a copper (I) salt.

Embodiment 39 provides the method of any one of Embodiments 36-38, wherein the (A) and 3,5-pyrandione are reacted in the presence of L-proline.

Embodiment 40 provides the method of any one of Embodiments 36-39, wherein the (A) and 3,5-pyrandione are reacted in the presence of a base.

Embodiment 41 provides the method of Embodiment 40, wherein the base comprises an alkali carbonate.

Embodiment 42 provides the method of any one of Embodiments 36-41, wherein the treating of the keto-acid intermediate with an acid comprises acidification of the keto-acid intermediate to a pH of about 2.

Embodiment 43 provides the method of any one of Embodiments 36-42, wherein the reaction of (A) and 3,5-pyrandione occurs at a temperature of about 70° C.

Embodiment 44 provides the method of any one of Embodiments 36-43, wherein (A) and the 3,5-pyrandione are reacted at a molar ratio of about 1:1.2.

The disclosures of each and every patent, patent application, and publication cited herein are hereby incorporated herein by reference in their entirety. While this disclosure has been disclosed with reference to specific embodiments, it is apparent that other embodiments and variations of this disclosure may be devised by others skilled in the art without departing from the true spirit and scope of the disclosure. The appended claims are intended to be construed to include all such embodiments and equivalent variations.

1. A method of preparing (S)—N-(8,9-difluoro-6-oxo-1,4,5,6-tetrahydro-2H-pyrano[3,4-c]isoquinolin-1-yl)-5,6-difluoro-N-methyl-1H-indole-2-carboxamide (X), or a salt or solvate thereof: 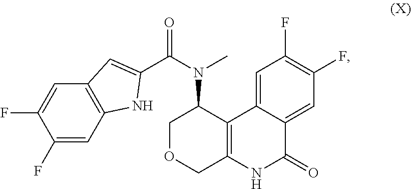 [SMILES: CN(C(=O)c1cc2cc(F)c(F)cc2[nH]1)[C@@H]1COCc2[nH]c(=O)c3cc(F)c(F)cc3c21]
the method comprising reacting (S)-8,9-difluoro-1-(methylamino)-1,5-dihydro-2H-pyrano[3,4-c]isoquinolin-6(4H)-one (F): 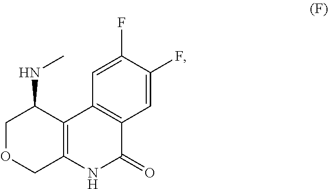 [SMILES: CN[C@@H]1COCc2[nH]c(=O)c3cc(F)c(F)cc3c21]
and 5,6-difluoro-1H-indole-2-carbonyl chloride (H): 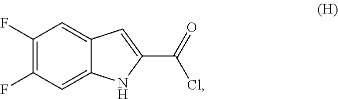 [SMILES: O=C(Cl)c1cc2cc(F)c(F)cc2[nH]1]
so as to generate a first reaction system comprising (X).

2. The method of claim 1, wherein the reaction of (F) and (H) is performed in the presence of a base.

3. The method of claim 2, wherein the base comprises K₂CO₃.

4. The method of claim 1, wherein (F) and (H) are reacted in a solvent comprising at least one of THF and H₂O.

5. The method of claim 1, wherein purification of (X) comprises adding at least one organic solvent to the first reaction system to generate a biphasic system.

6. The method of claim 5, wherein the organic solvent comprises ethyl acetate.

7. The method of claim 5, wherein the purification of (X) further comprises separating the biphasic system to provide an aqueous phase and an organic phase.

8. The method of claim 7, wherein the purification of (X) further comprises exchanging at least a fraction of the organic phase with ethanol to generate a first solution.

9. The method of claim 8, wherein the purification of (X) further comprises adding water to the first solution to provide a second solution.

10. The method of claim 9, wherein the purification of (X) further comprises heating and cooling the second solution to yield solid (X).

11. The method of claim 1, wherein (H) is prepared by reacting 5,6-difluoro-1H-indole-2-carboxylic acid (G) and a chlorinating agent:

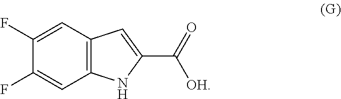 [SMILES: O=C(O)c1cc2cc(F)c(F)cc2[nH]1]

12. The method of claim 11, wherein the chlorinating agent is selected from the group consisting of (COCl)₂ and SOCl₂.

13. The method of claim 1, wherein (F) is prepared by reacting (S)-8,9-difluoro-1-(methyl((R)-1-phenylethyl)amino)-1,5-dihydro-2H-pyrano[3,4-c]isoquinolin-6(4H)-one (E) with hydrogen gas (H₂) in the presence of a hydrogenation catalyst and an acid:

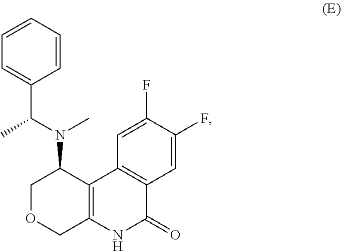 [SMILES: C[C@H](c1ccccc1)N(C)[C@@H]1COCc2[nH]c(=O)c3cc(F)c(F)cc3c21]

14. The method of claim 13, wherein the hydrogenation catalyst comprises palladium on carbon (Pd/C).

15. The method of claim 13, wherein the acid comprises trifluoroacetic acid (TFA).

16. The method of claim 13, wherein (E) and hydrogen gas are reacted in a solvent comprising methanol.

17. The method of claim 16, wherein purification of (F) comprises exchanging at least a fraction of the methanol with 2-methyltetrahydrofuran, thereby generating a first solution.

18. The method of claim 17, wherein at least partial concentration of the first solution yields solid (F).

19. The method of claim 13, wherein (E) is prepared by reductive alkylation of (S)-8,9-difluoro-1-(((R)-1-phenylethyl)amino)-1,5-dihydro-2H-pyrano[3,4-c]isoquinolin-6(4H)-one (D) with formaldehyde:

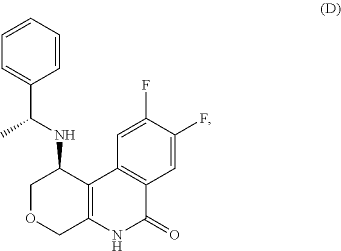 [SMILES: C[C@@H](N[C@@H]1COCc2[nH]c(=O)c3cc(F)c(F)cc3c21)c1ccccc1]

20. The method of claim 19, wherein the reductive alkylation of the (D) is performed in a solvent comprising 2-methyltetrahydrofuran.

21. The method of claim 19, wherein (D) is prepared by reacting (R)-1-phenylethan-1-amine: 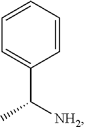
and 8,9-difluoro-2H-pyrano[3,4-c]isoquinoline-1,6(4H,5H)-dione (C): 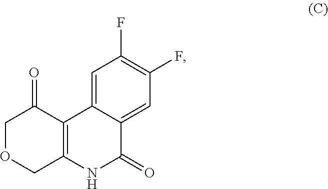 [SMILES: O=C1COCc2[nH]c(=O)c3cc(F)c(F)cc3c21]
so as to form an imine intermediate, and treating the imine intermediate with a reducing agent to form (D).

22. The method of claim 21, wherein (C) and (R)-1-phenylethan-1-amine are reacted in the presence of a titanium (IV) alkoxide to form the imine intermediate.

23. The method of claim 22, wherein the titanium (IV) alkoxide comprises Ti(OEt)₄.

24. The method of claim 21, wherein the reducing agent comprises a borohydride.

25. The method of claim 1, wherein (F) is prepared by reacting (S)-8,9-difluoro-1-(((R)-1-(4-methoxyphenyl)ethyl)(methyl)amino)-1,5-dihydro-2H-pyrano[3,4-c]isoquinolin-6(4H)-one (E′) with an acid:

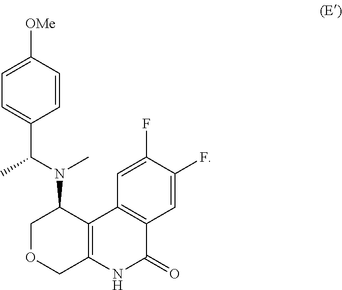 [SMILES: COc1ccc([C@@H](C)N(C)[C@@H]2COCc3[nH]c(=O)c4cc(F)c(F)cc4c32)cc1]

26. The method of claim 25, wherein the acid comprises trifluoroacetic acid (TFA).

27. The method of claim 2, wherein (E′) and the acid are reacted in the presence of a trialkylsilane.

28. The method of claim 25, wherein (E′) is prepared by reductive alkylation of (S)-8,9-difluoro-1-(((R)-1-(4-methoxyphenyl)ethyl)amino)-1,5-dihydro-2H-pyrano[3,4-c]isoquinolin-6(4H)-one (D′) with formaldehyde:

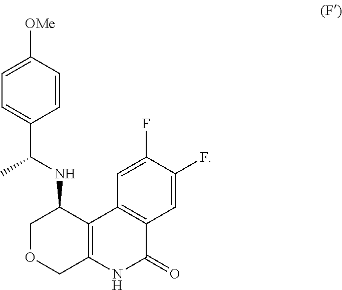 [SMILES: COc1ccc([C@@H](C)N[C@@H]2COCc3[nH]c(=O)c4cc(F)c(F)cc4c32)cc1]

29. The method of claim 28, wherein (D′) is prepared by reacting (R)-1-(4-methoxyphenyl)ethan-1-amine: 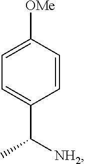
and 8,9-difluoro-2H-pyrano[3,4-c]isoquinoline-1,6(4H,5H)-dione (C): 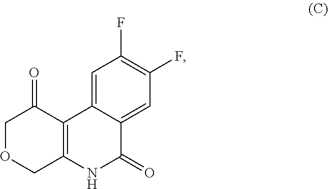 [SMILES: O=C1COCc2[nH]c(=O)c3cc(F)c(F)cc3c21]
so as to form an imine intermediate, and treating the imine intermediate with a reducing agent to form (D′).

30. The method of claim 29, wherein (C) and the (R)-1-(4-methoxyphenyl)ethan-1-amine are reacted in the presence of a titanium (IV) alkoxide to form the imine intermediate.

31. The method of claim 2, wherein the reducing agent comprises a borohydride.

32. The method of claim 21, wherein the (C) is prepared by reacting 8,9-difluoropyrano[3,4-c]isochromene-1,6(2H,4H)-dione (B): 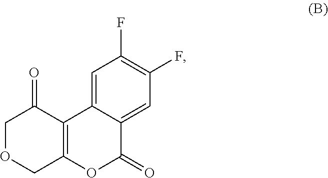 [SMILES: O=C1COCc2oc(=O)c3cc(F)c(F)cc3c21]
with an ammonium salt.

33. The method of claim 32, wherein the ammonium salt comprises ammonium hydroxide.

34. The method of claim 33, wherein the ammonium hydroxide comprises an aqueous solution.

35. The method of claim 32, wherein the reaction of (B) and the ammonium salt occurs at a temperature of about 50° C.

36. The method of claim 32, wherein (B) is prepared by reacting 2-bromo-4,5-difluorobenzoic acid (A): 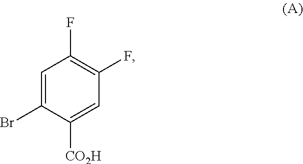 [SMILES: O=C(O)c1cc(F)c(F)cc1Br]
with 3,5-pyrandione so as to form a keto-acid intermediate, and treating the keto-acid intermediate with an acid to form (B).

37. The method of claim 36, wherein (A) and 3,5-pyrandione are reacted in the presence of a Lewis acid.

38. The method of claim 37, wherein the Lewis acid comprises a copper (I) salt.

39. The method of claim 36, wherein the (A) and 3,5-pyrandione are reacted in the presence of L-proline.

40. The method of claim 36, wherein the (A) and 3,5-pyrandione are reacted in the presence of a base.

41. The method of claim 40, wherein the base comprises an alkali carbonate.

42. The method of claim 36, wherein the treating of the keto-acid intermediate with an acid comprises acidification of the keto-acid intermediate to a pH of about 2.

43. The method of claim 36, wherein the reaction of (A) and 3,5-pyrandione occurs at a temperature of about 70° C.

44. The method of claim 36, wherein (A) and the 3,5-pyrandione are reacted at a molar ratio of about 1:1.2.
